(19) United States
(12) Patent Application Publication
(10) Pub. No.: US 2010/0216718 A1
(43) Pub. Date: Aug. 26, 2010

(54) Cancer Classification and Methods of Use

(75) Inventor: Klarisa Rikova, Reading, MA (US)

Correspondence Address: Nancy Chiu Wilker, Ph.D.;Chief Intellectual Property Counsel, CELL SIGNALING TECHNOLOGY, INC., 3 Trask Lane, Danvers, MA, 01923, US

(73) Assignee: CELL SIGNALING TECHNOLOGY, INC., Danvers, MA (US)

(21) Appl. No.: 12/738,524

(22) PCT Filed: Oct. 20, 2008
(86) PCT No.: PCT/US08/11969

§ 371 Date: Apr. 16, 2010

Related U.S. Application Data
(60) Provisional application No. 60/999,628, filed on Oct. 19, 2007.

(51) Int. Cl. A61K 38/16; G01N 33/574; A61P 35/00

(52) U.S. Cl. 514/12; 435/7.23

(57) ABSTRACT

The present invention relates to methods of classifying cancer cells based on the presence, absence or level of a tyrosine kinase or a phosphorylated tyrosine kinase. The present invention also relates to methods of treating cancer using cancer classification. The present invention further relates to methods of determining the effectiveness of a treatment for cancer using cancer classification.

### FIELD OF THE INVENTION

The present invention relates to methods of classifying cancer cells based on the presence, absence or level of a tyrosine kinase or a phosphorylated tyrosine kinase. The present invention also relates to methods of treating cancer using cancer classification. The present invention further relates to methods of determining the effectiveness of a treatment for cancer using cancer classification.

### BACKGROUND OF THE INVENTION

Lung cancer is the leading cause of cancer mortality in the world today. Despite decades of intensive analysis, the majority of molecular defects that play a causal role in the development of lung cancer remain unknown. Two oncogenes important in lung cancer are K-RAS and EGFR, mutated in 15% and 10% of NSCLC patients. Large-scale DNA sequencing efforts have identified mutations in PI3KCA, ERBB2, and B-RAF that together represent another 5% of NSCLC patients (Greenman, C., Stephens, P., Smith, R., Dalgliesh, G. L., Hunter, C., Bignell, G., Davies, H., Teague, J., Butler, A., Stevens, C., et al. (2007). Patterns of somatic mutation in human cancer genomes. Nature 446, 153-158; Thomas, R. K., Baker, A. C., Debiasi, R. M., Winckler, W., Laframboise, T., Lin, W. M., Wang, M., Feng, W., Zander, T., Macconnaill, L. E., et al. (2007). High-throughput oncogene mutation profiling in human cancer. Nat. Genet. 39, 347-351). Analysis of recurrent chromosomal aberrations including amplification and deletion using CGH and SNP arrays promises to identify many additional genes altered in cancer (Chin, K., DeVries, S., Fridlyand, J., Spellman, P. T., Roydasgupta, R., Kuo, W. L., Lapuk, A., Neve, R. M., Qian, Z., Ryder, T., et al. (2006). Genomic and transcriptional aberrations linked to breast cancer pathophysiologies. Cancer Cell 10, 529-541; Neve, R. M., Chin, K., Fridlyand, J., Yeh, J., Baehner, F. L., Fevr, T., Clark, L., Bayani, N., Coppe, J. P., Tong, F., et al. (2006). A collection of breast cancer cell lines for the study of functionally distinct cancer subtypes. Cancer Cell 10, 515-527). However, genetic approaches suffer from the difficulty of identifying a small number of causal changes within a sea of changes associated with genome instability. Thus, there remains a need for methods that focus on the key lesions driving disease.

One such strategy involves analysis of the cellular signaling pathways corrupted in cancer (Vogelstein, B., and Kinzler, K. W. (2004). Cancer genes and the pathways they control. Nat. Med. 10, 789-799). Signaling via tyrosine kinases is often deregulated in cancer as these enzymes mediate most growth and survival signaling in multicellular organisms (Blume-Jensen, P., and Hunter, T. (2001). Oncogenic kinase signalling. Nature 411, 355-365). Selective tyrosine kinase inhibitors have recently shown success in treating cancer. However, their success depends upon the identification of tumors that are driven by activated kinases and are therefore dependent upon the targeted kinase for their survival and clinical benefit (Dowell, J. E., and Minna, J. D. (2005). Chasing mutations in the epidermal growth factor in lung cancer. N. Engl. J. Med. 352, 830-832; Weinstein, I. B. (2002). Cancer. Addiction to oncogenes—the Achilles heal of cancer. Science 297, 63-64). Thus, there remains a need for methods to identify activated tyrosine kinases in the initiation and progression of disease.

### SUMMARY OF THE INVENTION

It has now been found that cancer cells can be classified based on aberrant tyrosine kinase. Such classification is useful in treating cancer and in determining the effectiveness of cancer treatment.

Accordingly, the present invention provides methods of classifying cancer cells in a sample based on the presence, absence, or levels of the one or more tyrosine kinases in at least one signaling pathway. The present invention also provides methods of classifying cancer cells based on the presence, absence, or levels of one or more phosphorylated tyrosine kinases in at least one signaling pathway.

In addition, the present invention provides methods of treating cancer in a subject by classifying cancer cells based on the levels of one or more aberrantly expressed tyrosine kinases in at least one signaling pathway and administering an effective dose of one or more tyrosine kinase inhibitors based on the classification. The present invention also provides methods of treating cancer by classifying cancer cells based on the levels of one or more aberrantly phosphorylated tyrosine kinases in at least one signaling pathway and administering an effective dose of one or more tyrosine kinase inhibitors based on the classification.

The present invention further provides methods of determining the effectiveness of a treatment for cancer in a subject, based on detecting the presence, absence, or levels of one or more tyrosine kinases in at least one signaling pathway in a sample, wherein the presence, absence, or levels of the one or more tyrosine kinases is correlated to the effectiveness of the treatment. The present invention also provides methods of determining the effectiveness of a treatment for cancer, based on detecting the presence, absence, or levels of one or more phosphorylated tyrosine kinases in at least one signaling pathway in a sample, wherein the presence, absence, or levels of the one or more tyrosine kinases is correlated to the effectiveness of the treatment.

In some embodiments, the presence, absence, or levels of the one or more tyrosine kinases is determined using one or more of FISH, INC, PCR, MS, flow cytometry, Western blotting, or ELISA.

In some embodiments, the presence, absence, or levels of one or more phosphorylated tyrosine kinases is determined by immunoprecipitating phosphopeptides and analyzing the immunoprecipitated phosphopeptides.

In some embodiments, the tyrosine kinases is selected from EGFR, FAK, Src, ALK, PDGFRa, Erb2, ROS, cMet, Ax1, ephA2, DDR1, DDR2, or FGFR.

In some embodiments, the cancer cells are classified using one or more statistical methods. In some aspects of this embodiment, the statistical method is unsupervised Pearson clustering.

In some embodiments, the cancer cells are classified as having only one or two highly phosphorylated tyrosine kinases. In other embodiments, the cancer cells are classified as expressing phosphorylated Fak, Src, Ax1, and at least one receptor tyrosine kinase selected from the group consisting of EGFR, ALK, PDGFRa, Erb2, ROS, cMet, Ax1, ephA2, DDR1, DDR2, FGFR, VEGR-2, IGFR1, LYN, HCK, HER2, IRS1, IRS2, BRK, EphB4, FGFR1, ErbB3, VEGFR-1, EphB1, EphA4, EphA1, EphA5, Tyro3, EphB2, IGF1R, EphA2, EphB3, Mer, EphB4, and Kit. In other embodiments, the cancer cells are classified as expressing phosphorylated DDR1, Src, and Abl. In other embodiments, the cancer cells are classified as expressing phosphorylated Src and at least one receptor tyrosine kinases selected from the group consisting of EGFR, ALK, PDGFRa, Erb2, ROS, cMet, Ax1, ephA2, DDR1, DDR2, FGFR, VEGR-2, IGFR1, LYN, HCK, HER2, IRS1, IRS2, BRK, EphB4, FGFR1, ErbB3, VEGFR-1, EphB1, EphA4, EphA1, EphA5, Tyro3, EphB2, IGFIR, EphA2, EphB3, Mer, EphB4, and Kit. In other embodiments, the cancer cells are classified as expressing phosphorylated Src and Abl.

In some embodiments, the cancer cells are from lung cancer, hematological cancer, prostate cancer, breast cancer, or tumor of the gastrointestinal tract. In some embodiments, the methods are used to classify non-small cell lung cancers (NSCLCs).

### BRIEF DESCRIPTION OF THE FIGURES

FIG. 1A is micrographs of IHC staining of paraffin-embedded human NSCLC tumor tissues showing high, medium, and low phosphotyrosine expression.

FIG. 1B is a Western blot showing phosphotyrosine signaling in 22 different NSCLC cell lines showing different patterns of phosphotyrosine reactivity.

FIG. 1C is a diagram showing an embodiment of immunoaffinity profiling method. Cells or tissues are lysed in urea buffer and digested with protease. The resulting peptides are immunoaffinity purified using immobilized phosphotyrosine-specific antibody (P-Tyr-100) and analyzed by LC-MS/MS. Because larger liquid chromatography peaks are sampled more times than are smaller peaks, the number of observed spectra assigned to a particular protein is a semiquantitative measure of the abundance of that protein.

FIG. 1D is a Western blot showing Met and Phospho-Met(Tyr1234/5) expression in NSCLC cell lines. Shown below is a comparison of the number of phosphopeptides identified by MS/MS with the immunoblotting. The number of different sites identified are shown in parenthesis.

FIG. 2A is pie charts showing distribution of phosphoprotein types. Each observed phosphoprotein was assigned a protein category from the PhosphoSite ontology. The numbers of unique proteins in each category, as a fraction of the total, are represented by the wedges of the pies.

FIG. 2B is pie charts showing distribution of spectral counts among receptor tyrosine kinases (RTK). The total numbers of observed spectra assigned to each RTK over all of the cell lines (top) or the tumors (bottom) are represented as fractions of the total RTK spectra observed.

FIG. 2C are pie charts showing distribution of spectral counts among nonreceptor tyrosine kinases. The total numbers of observed spectra assigned to each TK (nonreceptor) over all of the cell lines (top) or the tumors (bottom) are represented as fractions of the total TK (nonreceptor) spectra observed.

FIGS. 2D and 2E are graphs showing phosphorylation of tyrosine kinases in lung cancer cell lines. The total number of boserved spectra assigned to each TK in each cell line was used as the basis for clustering using the Pearson correlation distance metric and average linkage. In FIG. 2D, no normalization has been applied. In FIG. 2E, each value in a row has had the row average subtracted.

FIG. 3A is a graph showing clustering of tumors by tyrosine phosphorylation. Spectral counts for tyrosine kinases in patient tumors were normalized to the count for GSK3β and then clustered as described in FIG. 2E. Clustering produced five groups of tumors with different sets of tyrosine kinases predominating.

FIGS. 3B-3D are graphs showing phosphorylation of selected nonkinase proteins in different tumor groups. Tumor samples were divided into the groups defined by the clustering in FIG. 3A, and spectral counts were normalized to the count for GSK3β. After all kinases were removed from the protein set, the data were clustered as in FIG. 2E and the top 30 proteins displayed. The tumors used in FIG. 3B were from group 1 in FIG. 3A, those in FIG. 3C from group 2, and those in FIG. 3D from group 4.

FIGS. 3E-3G are graphs showing most prominent phosphoproteins. Proteins were ranked, based on spectral counts, and the top 25 are shown. Before ranking the tumor proteins, each protein's counts were normalized to those for GSK3P, then the average count for that protein over all tumors was subtracted. Cell line proteins had their average count over all cell lines subtracted. Arrows indicate proteins shared between cell lines and tumors.

FIGS. 4A and 4B are pie charts showing distribution of spectral counts among receptor tyrosine kinases in H2228 and HCC78 cell lines. The total numbers of observed spectra assigned to each RTK are represented as fractions of the total RTK spectra observed.

FIG. 4C is a schematic representation of the EML4, ALK, and EML4-ALK fusion proteins. Arrow indicates the chromosomal breakpoint.

FIG. 4D is a schematic representation of the TFG, ALK, and TFG-ALK fusion proteins. Arrow indicates the chromosomal breakpoint.

FIG. 4E is a schematic representation of the SLC34A2, ROS, and SLC34A2-ROS fusion proteins. Arrow indicates the chromosomal breakpoint.

FIG. 4F is a schematic representation of the CD74, ROS, and CD74-ROS fusion proteins. Arrow indicates the chromosomal breakpoint.

FIG. 5A is a pie chart showing distribution of spectral counts among receptor tyrosine kinases in H1703.

FIG. 5B is Western blots showing the effects of EGFR and PDGFR inhibitors on Akt phosphorylation. H1703 cells were either untreated or treated with EGF, EGF with Iressa, or Gleevec for 1 hr, and the levels of EGFR, PDGFRa, Akt were determined by western blot. Phosphorylation of EGFR(Tyr1068) and Akt(Ser473) were determined using phosphorylation-state-specific antibodies.

FIG. 5C is a graph showing that Imatinib mesylate inhibits cell growth and induces apoptosis in H1703 cells. H1703 cells were treated with Gleevec for 72 hr, and MTS assay was performed. Results from the means of triplicate experiments (error bars indicate standard deviations) were shown.

FIG. 5D is a graph showing treatment of Imatinib on H1703 mouse xenographs. Mice with similar tumor size were divided to two groups, one group (5 mice) was treated with Gleevec, the other group (5 mice) was not treated. After 7 days of treatment, the size (mm length×mm width) of each tumor was measured.

FIG. 5E is a cartoon showing regulation of PDGFRα phosphorylation in H1703 cells by Imatinib. H1703 cell were labeled with light and heavy amino acids and analyzed by LC-MS/MS tandem mass spectrometry as described for SILAC. PDGFRα phosphorylation sites detected by mass spectrometry were indicated as well as the fold change measured after a 3 hr treatment with Imatinib.

FIG. 5F is a cartoon showing regulation of PDGFRα downstream signaling in H1703 cells as determined by SILAC and LC-MS/MS. Red circles depict proteins with decreased phosphorylation following Imatinib treatment. Black and red arrows indicate known and predicted (scansite and netphosK) substrates, respectively.

FIG. 6 is a graph showing clustering of phosphorylation sites on tyrosine kinases. For each tumor sample, the average count for the site across all samples was subtracted. The samples were then clustered using the 120 sites with the highest standard deviation across all samples, with the Pearson correlation distance metric, and average linkage.

FIG. 7 is a T-Test comparison showing signaling difference between tumor and adjacent tissues. Spectral counts for each protein in tumor and adjacent tissues were normalized to the count for GSK3 beta. Average counts across adjacent tissues were subtracted from all tumors and adjacent tissues. T-Test was carried out using TIGR's MeV program (Saeed, A. I., Sharov, V., White, J., Li, J., Liang, W., Bhagabati, N., Braisted, J., Klapa, M., Currier, T., Thiagarajan, M., et al. (2003) TM4: a free, open-source system for microarray data management and analysis. Biotechniques 34, 374-378) with Pearson Correlation Distance and Average linkage clustering to identify tyrosine phosphorylated proteins that showed a significant difference between adjacent and tumor tissue.

FIG. 8A is a Western blot showing ALK expression in NSCLC cell lines. ALK expression is highly restricted to H2228 cell.

FIG. 8B is a Western blot showing ROS expression in NSCLC cell lines. ROS expression is highly restricted to HCC78 cell line.

FIGS. 8C and 8D are a bar graph and Western blots, respectively, showing that knock down of ROS inhibits cell growth and induces cell death in HCC78 cells. HCC78 and H2066 cells were transfected with siRNA for ROS for 48 hrs. The viability of control and transfected cells was determined by the Trypan blue exclusion method. The mean percentage (of 4 experiments)+/−SD of viable cells is represented as bar graphs. The cell lysates from both control siRNA and ROS siRNA (100 nM) were immunoblotted with ROS, Cleaved-PARP, and β-actin antibodies.

FIG. 8E is a bar graph and a Western blot showing an in vitro kinase assay. pExchange-2 or pExchange-2/SLC34A2-ROS(S) vector was transiently transfected into 293T cells, ROS fusion protein was immunoprecipitated with Myc-tag antibody, and kinase assay was performed.

FIG. 8F is Western blots showing subcellular localization of ROS fusion protein. pExchange-2 or pExchange-2/SLC34A2-ROS(S) vector was transiently transfected into 293T cells. Subcellular localization of the fusion protein was detected with Myc-tag antibody. IGF1R, β-actin, and lamin A/C were used as a marker for plasma membrane (PM), Cytosol, and Nuclei fraction.

FIG. 8G is a diagram and micrographs showing that the ALK break-apart rearrangement probe contains two differently labeled probes on opposite sides of the breakpoint of the ALK gene. When hybridized, the native ALK region appears as an orange/green (yellow) fusion signal, while rearrangement at this locus will result in separate orange and green signals. The H2228 cell line and a patient sample contain two normal copies of ALK (yellow) and one proximal probe (red; white arrow) from the 3′ part of the ALK locus. The 5′ part of the locus appears to be deleted. Schematic representation of the EML4, ALK and EML4-ALK fusion proteins. Arrow indicates the chromosomal breakpoint.

FIG. 8H is a diagram and micrographs showing rearrangement within the ROS locus. A break-apart probe was used to analyze rearrangement within the ROS locus. Translocation within the ROS locus leads to separation of yellow signals into red or green signals (white arrows) shown in cell line HCC78 (left) and an NSCLC adenocarcinoma sample (right).

FIG. 9A is a Western blot showing PDGFRα in NSCLC cell lines. PDGFRα expression is highly restricted to H1703 cell line.

FIG. 9B is Western blots showing dose-dependent inhibition of PDGFR α and Akt phosphorylation by Imatinib mesylate (Gleevec) in H1703 cells. H1703 cells were treated with the indicated amount of Imatinib mesylate for 1 hour and the levels of Phospho-PDGFRα (Tyr754), phospho-Akt (Ser473), and phospho-MAPK (Thr202/Tyr204) measured by Western blot. The total protein levels of PDGFRα, Akt, and MAPK were also determined in the same samples.

FIG. 9C is a bar graph showing results of an apoptosis assay. Imatinib mesylate (1 μM, 10 μM) or DMSO (control) was added to 40% confluent H1703 cells, 24 hours later both adhering cells and floating cells were harvested, and apoptosis was measured by quantifying cleaved caspase-3 by flow cytometry. Results from the mean of 3 independent experiments are shown (error bars indicate standard deviations).

FIG. 9D is Western blots showing that Imatinib induces cleaved PARP expression in H1703 cells. H1703 cells were treated with increasing concentrations of Gleevec for 3 hours and cleaved-PARP measured by immunoblotting. PDGFR alpha levels were measured to control for total protein loading.

FIG. 9E is Western blots that confirm gleevec sensitive phosphorylation sites. Western analysis using site and phosphorylation-specific antibodies confirms decreased phosphorylation of PDGFRα, PLCγ1, and SHP2 by Gleevec at the same sites identified by mass spectrometry and under the same Imatinib treatment conditions (1 μM for 3 hours). Phosphorylation of Stat3, as predicted by mass spectrometry, was not changed.

FIG. 9F is pictures showing that Imatinib mesylate blocks tumor growth in mouse xenographs prepared from H1703 cells. Typical tumor size from 3 untreated mice (red arrow) and 3 Gleevec treated mice (blue arrow) after 7 days of Imatinib treatment at 50 mg/kg.

FIG. 9G is micrographs showing that PDGFRa expression was seen more frequently in adenocarcinoma and Bronchioloalveolar Carcinoma.

FIG. 9H is a diagram and micrographs showing amplification of PDGFRα. A normal control samples is shown on the left. Red signals indicate the PDGFRα probe (white arrow) and green signals the centromere, located on chromosome 4 in close proximity to PDGFRα. Amplification of PDGFRα in interphase nuclei from a squamous cell carcinoma patient is shown on the right. The large amplification is marked with a yellow arrow. This cell has 3 copies of chromosome 4 of which one shows amplification in the PDGFRα locus.

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

In order to further define the invention, the following terms and definitions are provided herein.

The term “sample” refers to a specimen that is obtained as or isolated from tumor tissue, brain tissue, cerebrospinal fluid, blood, plasma, serum, lymph, lymph nodes, spleen, liver, bone marrow, or any other biological specimen containing cancer cells.

The term “treating” or “treatment” is intended to mean reversing, mitigating, inhibiting the progress of, preventing or alleviating the symptoms of cancer in a mammal or the improvement of an ascertainable measurement associated with that cancer.

The term “subject” refers to a mammal, including, but not limited to, human, primate, equine, avian, bovine, porcine, canine, feline and murine.

The term “an effective dose” refers to the amount of an inhibitor sufficient to inhibit a tyrosine kinase.

The term “effectiveness of a treatment” refers the degree to which a disorder or condition, or one or more symptoms thereof, is reversed, alleviated, or prevented by a treatment, or the degree to which the progress of a disorder or condition is inhibited.

### Methods of Classifying Cancer Cells

The present invention provides methods of classifying cancer cells in a sample. In some embodiments, the methods comprise the steps of obtaining a sample of cancer cells; detecting the presence, absence, or levels of one or more tyrosine kinases in at least one signaling pathway in the sample; and classifying the cancer cells based on the presence, absence, or levels of the one or more tyrosine kinases. In alternate embodiments, the methods comprise the steps of obtaining a sample of cancer cells; detecting the presence, absence, or levels of one or more phosphorylated tyrosine kinases in at least one signaling pathway in the sample; and classifying the cancer cells based on the presence, absence, or levels of the one or more phosphorylated tyrosine kinases.

Cancer cells that may be used in the methods of the present invention include, but are not limited to, those cells derived from a cancer cell line or a solid tumor within a subject. Cancer cells may be obtained from any type of cancer, including, but not limited to, lung cancer (including squamous cell carcinoma of the lung), hematological cancer (including lymphoma), prostate cancer, breast cancer, and tumor of the gastrointestinal tract. In some embodiments, the cancer is lung cell. In preferred embodiments, the cancer is nonsmall cell lung cancer.

As used herein, the term tyrosine kinases generally refers to non-receptor tyrosine kinases and receptor tyrosine kinases. Non-receptor tyrosine kinases include, but are not limited to, ABL, ACK, CSK, FAK, FES, FRK, JAK, SRC, TEC, and SYK. Receptor tyrosine kinases include, but are not limited to, ALK, AXL, DDR1, DDR2, EGFR, EPH, ERB2, FGFR, INSR, MET, MUSK, PDGFR, PTK7, RET, ROR, ROS, TYK, TIE, TRK, VEGFR, AATYK, ephA2, VEGR-2, IGFR1, LYN, HCK, HER2, IRS1, IRS2, BRK, EphB4, FGFR1, ErbB3, EphB1, EphA4, EphA1, EphA5, Tyro3, EphB2, IGF1R, EphA2, EphB3, Mer, EphB4, and Kit. See Robinson, Wu and Lin, 2000, the entire content of which is incorporated by reference.

According to one embodiment, the cancer cells in a sample are classified based on detecting the presence, absence, or levels of tyrosine kinases. Suitable detection methods are well known to those skilled in the art and include, but are not limited to, florescent in situ hybridization (FISH), immunohistochemistry polymerase chain reaction (PCR), mass spectrometry (MS), flow cytometry, Western blotting, and enzyme-linked immunoadsorbent assay (ELISA).

According to another embodiment, the cancer cells in a sample are classified based on detecting the presence, absence, or levels of phosphorylated tyrosine kinases. Suitable detection methods are well known to those skilled in the art and include, but are not limited to, immunoprecipitation of phosphopeptides from a sample and analysis of the immunoprecipitated phosphopeptides using, e.g., liquid chromatography (LC) MS/MS.

According to yet another embodiment, cancer cells in a sample are classified based on detecting the presence, absence, or levels of the activity of one or more tyrosine kinases in at least one signaling pathway in the sample. Suitable detection methods are well known to those skilled in the art and include, but are not limited to, those disclosed in U. S. Pat. Nos. 6,066,462, 6,348,310, and 6,753,157, and European Patent No. 0 760 678 B9, the entire content of each of which are incorporated herein by reference.

In some embodiments, the classification step is performed without the aid of any statistical or computational method. This embodiment is preferred when the number of samples or the number of tyrosine kinases to be examined are small.

In other embodiments, classification step is performed with the aid of statistical or computational methods. This embodiment is preferred when the number of samples or the number of tyrosine kinases to be examined are large. Statistical methods are known to persons of ordinary skill in the art and include, but are not limited to, computer programs. Suitable computer programs, include, but are not limited to, unsupervised Pearson clustering.

In some embodiments, the cancer cells are classified as having only one or two highly phosphorylated tyrosine kinases (class I). In other embodiments, the cancer cells are classified as expressing phosphorylated Fak, Src, Abl, and at least one receptor tyrosine kinase selected from the group consisting of EGFR, ALK, PDGFRa, Erb2, ROS, cMet, Ax1, ephA2, DDR1, DDR2, FGFR, VEGR-2, IGFR1, LYN, HCK, HER2, IRS1, IRS2 and BRK (class II). In other embodiments, the cancer cells are classified as expressing phosphorylated DDR1, Src, and Abl (class III). In other embodiments, the cancer cells are classified as expressing phosphorylated Src and at least one receptor tyrosine kinases selected from the group consisting of EGFR, ALK, PDGFRa, Erb2, ROS, cMet, Ax1, ephA2, DDR1, DDR2, FGFR, VEGR-2, IGFR1, LYN, HCK, HER2, IRS1, IRS2 and BRK (class IV). In other embodiments, the cancer cells are classified as expressing phosphorylated Src and Abl (class V).

In a preferred embodiment, the present invention provides methods to classify nonsmall cell lung cancer cells. According to one aspect of this embodiment, the method comprises obtaining a sample of NSCLC cells; determining the presence, absence, or levels of one or more tyrosine kinases in at least one signaling pathway in the sample; and classifying the NSCLC cells based on the presence, absence, or levels of the one or more tyrosine kinases. According to another aspect of this embodiment, the method comprises obtaining a sample of NSCLC cells; determining the presence, absence, or levels of one or more phosphorylated tyrosine kinases in at least one signaling pathway in the sample; and classifying the NSCLC cells based on the presence, absence, or levels of one or more phosphorylated tyrosine kinases.

### Methods of Treating Cancer

The present invention also provides a method of treating cancer in a subject. In some embodiments, the method comprises the steps of obtaining a sample of cancer cells from the subject; classifying the cancer cells based on the levels of one or more aberrantly expressed tyrosine kinases in at least one signaling pathway in the sample; and administering an effective dose of one or more tyrosine kinase inhibitors based on the classification. In alternate embodiments, the method comprises the steps of obtaining a sample of cancer cells from the subject; classifying the cancer cells based on the levels of one or more aberrantly phosphorylated tyrosine kinases in at least one signaling pathway in the sample; and administering an effective dose of one or more tyrosine kinase inhibitors based on the classification.

The cancer cells that may be used in this method include, but are not limited to, those derived from lung cancer (including squamous cell carcinoma of the lung), hematological cancer (including lymphoma), prostate cancer, breast cancer, and tumor of the gastrointestinal tract. In some embodiments, the cancer is lung cell. In preferred embodiments, the cancer is nonsmall cell lung cancer.

The sample of cancer cells may be obtained by any method known in the art, including but not limited to, obtaining a specimen of a tumor from a subject.

In some embodiments, the cancer cells are classified based on aberrantly expressed tyrosine kinase. In alternate embodiments, the cancer cells are classified based on aberrantly expressed phosphorylated tyrosine kinase. According to these embodiments, the expression or phosphorylation levels or activities of the tyrosine kinases (or phosphorylated tyrosine kinases) are detected and compared with those detected in samples containing normal cells.

In some embodiments, the cancer cells are classified as having only one or two highly phosphorylated tyrosine kinases (class I). In other embodiments, the cancer cells are classified as expressing phosphorylated Fak, Src, Abl, and at least one receptor tyrosine kinase selected from the group consisting of EGFR, ALK, PDGFRa, Erb2, ROS, cMet, Ax1, ephA2, DDR1, DDR2, FGFR, VEGR-2, IGFR1, LYN, HCK, HER2, IRS1, IRS2 and BRK (class II). In other embodiments, the cancer cells are classified as expressing phosphorylated DDR1, Src, and Ax1 (class III). In other embodiments, the cancer cells are classified as expressing phosphorylated Src and at least one receptor tyrosine kinases selected from the group consisting of EGFR, ALK, PDGFRa, Erb2, ROS, cMet, Ax1, ephA2, DDR1, DDR2, FGFR, VEGR-2, IGFR1, LYN, HCK, HER2, IRS1, IRS2 and BRK (class IV). In other embodiments, the cancer cells are classified as expressing phosphorylated Src and Abl (class V).

In the methods of treating cancer, an effective dose of one or more tyrosine kinase inhibitors is administered to a subject based on the classification. Suitable tyrosine kinase inhibitors that may be administered in the methods of the present invention are known in the art, and include, but are not limited to, Axitinib (also known as AG013736; Rugo, H. S., Herbst, R. S., Liu, G., Park, J. W., Kies, M. S., Steinfeldt, H. M., Pithavala, Y. K., Reich, S. D., Freddo, J. L., and Wilding, G. (2005) Phase I Trial of the Oral Antiangiogenesis Agent AG-013736 in Patients With Advanced Solid Tumors: Pharmacokinetic and Clinical Results. Journal of Clinical Oncology 23, 5474-5483), Bosutinib (Gambacorti-Passerini, C., Kantarjian, H. M., Baccarani, M., Porkka, K., Turkina, A., Zaritskey, A. Y., Agarwal, S., Hewes, B., and Khoury, H. J. (2008) Activity and tolerance of bosutinib in patients with AP and BP CML and Ph+ ALL. J. Clin. Oncol. 26 (May 20 suppl; abstr 7049)), Cediranib (also known as AZD2171; Wedge, S. R., Kendrew, J., Hennequin, L. F., Valentine, P. J., Barry, S. T., Brave, S. R., Smith, N. R., James, N. H., Dukes, M., Curwen, J. O., Chester, R., Jackson, J. A., Boffey, S. J., Kilburn, L. L., Barnett, S., Richmond, G. H. P., Wadsworth, P. F., Walker, M., Bigley, A. L., Taylor, S. T., Cooper, L., Beck, S., Jürgensmeier, J. M., and Ogilvie, D. J. (2005) AZD2171: A Highly Potent, Orally Bioavailable, Vascular Endothelial Growth Factor Receptor-2 Tyrosine Kinase Inhibitor for the Treatment of Cancer. Cancer Res. 65, 4389-4400), Dasatinib (Talpaz, M., Shah, N. P., Kantarjian, H., Donato, N., Nicoll, J., Paquette, R., Cortes, J., O'Brien, S., Nicaise, C., Bleickardt, E., Blackwood-Chirchir, M. A., Iyer, V., Chen, T.-T., Phil., Huang, F., Decillis, A. P., and Sawyers, C. L. (2006) Dasatinib in Imatinib-Resistant Philadelphia Chromosome—Positive Leukemias. N. Eng. J. Med. 354, 2531-2541), Erlotinib (Pérez-Soler, R., Chachoua, A., Hammond, L. A., Rowinsky, E. K., Huberman, M. Karp, D., Rigas, J., Clark, G. M., Santábarbara, P., and Bonomi, P. (2004) Determinants of Tumor Response and Survival With Erlotinib in Patients With Non-Small-Cell Lung Cancer. Journal of Clinical Oncology 22, 3238-3247.

Rappsilber, J., Ishihama, Y., and Mann, M. (2003) Stop and go extraction tips for matrix-assisted laser desorption/ionization, nanoelectrospray, and LC/MS sample pretreatment in proteomics. Anal Chem. 75(3):663-70.), Gefitinib (Pao, W., Miller, V., Zakowski, M., Doherty, J., Politi, K., Sarkaria, I., Singh, B., Heelan, R., Rusch, V., Fulton, L., et al. (2004). EGF receptor gene mutations are common in lung cancers from “never smokers” and are associated with sensitivity of tumors to gefitinib and erlotinib. Proc. Natl. Acad. Sci. USA 101, 13306-13311. Peduto, L., Reuter, V. E., Shaffer, D. R., Scher, H. I., and Blobel, C. P. (2005). Critical function for ADAM9 in mouse prostate cancer. Cancer Res. 65, 9312-9319), Imatinib (Deininger, M. W. N. and Druker B. J. (2003) Specific Targeted Therapy of Chronic Myelogenous Leukemia with Imatinib. Pharmacological Reviews 55, 401-423), Lapatinib (Burris III, H. A. (2004) Dual kinase inhibition in the treatment of breast cancer: initial experience with the EGFR/ErbB-2 inhibitor Lapatinib. The Ongologist 9 (suppl 3), 10-15), Lestaurtinib (Cephalon, Frazer, P A), Nilotinib (Kantarjian, H., Giles, F., Wunderle, L., Bhalla, K., O'Brien, S., Wassmann, B., Tanaka, C., Manley, P., Rae, P., Mietlowski, W., Bochinski, K., Hochhaus, A., Griffin, J. D., Hoelzer, D., Albitar, M., Dugan, M., Cortes, J., Alland, L., and Ottmann, O. G. (2006) Nilotinib in Imatinib-Resistant CML and Philadelphia Chromosome-Positive ALL. N. Eng. J. Med. 354, 2542-2551), Samaxanib (O'Donnell, A., Padhani, A., Hayes, C., Kakkar, A. J., Leach, M., Trigo, J. M., Scurr, M., Raynaud, F., and Phillips, S. (2005) A Phase I study of the angiogenesis inhibitor SU5416 (semaxanib) in solid tumours, incorporating dynamic contrast MR pharmacodynamic end points. British Journal of Cancer 93, 876-883), Sunitinib (Motzer, R. J., Hutson, T. E., Tomczak, P., Michaelson, M. D., Bukowski, R. M., Rixe, O., Oudard, S., Negrier, S., Szczylik, C., Kim, S. T., Chen, I., Bycott, P. W., Baum, C. M., and Figlin, R. A. (2007) Sunitinib versus Interferon Alfa in Metastatic Renal-Cell Carcinoma. N. Eng. J. Med. 356, 115-124), and Vandetanib (AstraZeneca, London, England).

The tyrosine kinase inhibitor may be administered using any of the various methods known in the art. In some embodiments, the tyrosine kinase inhibitor is administered intravenously. In some embodiments, the tyrosine kinase inhibitor is administered intramuscularly. In some embodiments, the tyrosine kinase inhibitor is administered subcutaneously.

### Methods of Determining Effectiveness of a Treatment

The present invention further provides methods of determining the effectiveness of a treatment for cancer in a subject. In some embodiments, the method comprises obtaining a sample of cancer cells from a subject; and detecting the presence, absence, or levels of one or more tyrosine kinases in at least one signaling pathway in the sample; wherein the presence, absence, or levels of the one or more tyrosine kinases is correlated to the effectiveness of the treatment. In other embodiments, the method comprises obtaining a sample of cancer cells from a subject; and detecting the presence, absence, or levels of one or more phosphorylated tyrosine kinases in at least one signaling pathway in the sample; wherein the presence, absence, or levels of the one or more tyrosine kinases is correlated to the effectiveness of the treatment.

The cancer cells that may be used in this method include, but are not limited to, those derived from lung cancer (including squamous cell carcinoma of the lung), hematological cancer (including lymphoma), prostate cancer, breast cancer, and tumor of the gastrointestinal tract. In some embodiments, the cancer is lung cell. In preferred embodiments, the cancer is nonsmall cell lung cancer.

In some embodiments, the presence, absence or levels of one or more tyrosine kinases is detected. In other embodiments, the presence, absence or levels of one or more phosphorylated tyrosine kinases is detected. Suitable methods for detecting tyrosine kinase include, but are not limited to, FISH, IHC, PCR, MS, flow cytometry, Western blotting, and ELISA. Suitable methods for detecting phosphorylated tyrosine kinase are well known in the art (e.g. U. S. Pat. Nos. 7,198,896 and 7,300,753 both of which are incorporated herein by reference in their entirety).

Without wishing to be bound by any theory, it is believed that, because protein tyrosine phosphorylations exhibit significant differences between cancer cells and normal cells, and among different cancer cells, the presence, absence, or levels of tyrosine kinases or phosphorylated tyrosine kinases in signaling pathways in different cancer cells may be indicators of the severity, stage, or type of cancers, thus correlating with the effectiveness of a cancer treatment.

In order that this invention be more fully understood, the following examples are set forth. These examples are for the purpose of illustration only and are not to be construed as limiting the scope of the invention in any way.

### EXAMPLES

### Example 1

### Phosphotyrosine Profiles of NSCLC Tumors and Cell Lines

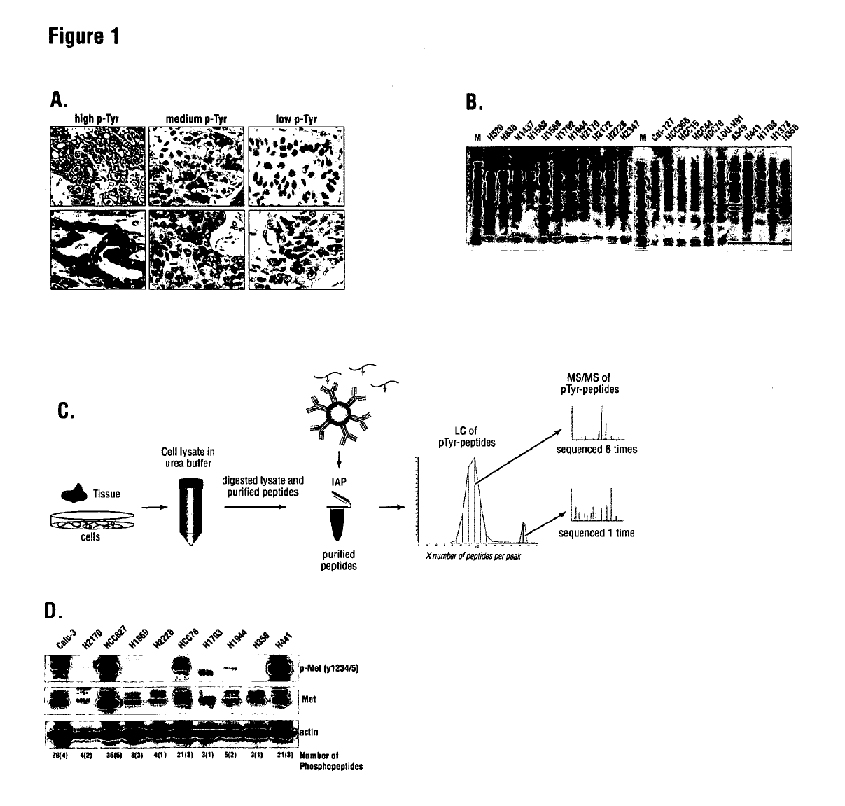

We used immunohistochemistry (IHC) and a phosphotyrosine-specific antibody to screen 96 paraffin-embedded, formalin-fixed tissue samples from NSCLC patients (FIG. 1A). Approximately 30% of tumors showed high levels of phosphotyrosine expression. This group of patient samples also showed high levels of receptor tyrosine kinase (RTK) expression, suggesting that RTK activity may play a role in the genesis of these lung tumors. Immunoblotting of 41 NSCLC cell lines with a phosphotyrosine specific antibody also showed heterogeneous reactivity especially in the molecular weight range characteristic of receptor tyrosine kinases (FIG. 1B).

To further characterize tyrosine kinase activity in NSCLC cell lines and solid tumors, we used an immunoaffinity phosphoproteomic approach. Because phosphotyrosine represents less than 1% of the cellular phosphoproteome as determined by tandem mass spectrometry (MS/MS) (Olsen, J. V., Blagoev, B., Gnad, F., Macek, B., Kumar, C., Mortensen, P., and Mann, M. (2006). Global, in vivo, and site-specific phosphorylation dynamics in signaling networks. Cell 127, 635-648) and is difficult to analyze by conventional methods, we used immunoaffinity purification with a phosphotyrosine antibody to enrich for phosphotyrosine-containing peptides prior to analysis by tandem mass spectrometry (Rush, J., Moritz, A., Lee, K. A., Guo, A., Goss, V. L., Spek, E. J., Zhang, H., Zha, X. M., Polakiewicz, R. D., and Comb, M. J. (2005). Immunoaffinity profiling of tyrosine phosphorylation in cancer cells. Nat. Biotechnol. 23, 94-101). All tumors were identified as NSCLC based upon standard pathology. Only tumors with greater than 50% of cancer cells were included in the analysis. We grew NSCLC cell lines overnight in low serum before analysis to reduce background phosphorylation resulting from culture conditions.

We detected phosphorylation status of a large number of sites (ranging between 150 and 1200 nonredundant sites/cell line or tumor) using this method and obtained phosphotyrosine profiles from a total of 41 NSCLC cell lines and 150 NSCLC tumors. 4551 sites of tyrosine phosphorylation were identified on greater than 2700 different proteins, dramatically extending our knowledge of tyrosine kinase signaling in NSCLC. We queried these sites against PhosphoSite (www.phosphosite.org), a comprehensive resource of known phosphorylation sites (Hornbeck, P. V., Chabra, I., Kornhauser, J. M., Skrzypek, E., and Zhang, B. (2004). PhosphoSite: A bioinformatics resource dedicated to physiological protein phosphorylation. Proteomics 4, 1551-1561) and found that more than 85% appeared novel. These data have been deposited in PhosphoSite and the data sets are freely available via http://www.phosphosite.org/napers/rikova01.html.

### Example 2

### NSCLC Tyrosine Phosphorylation

As an initial step to screen for phosphotyrosine signaling abnormalities and to compare NSCLC proteins based upon phosphopeptide data sets, we adopted a semiquantitative approach using the number of phosphopeptide assignments to approximate the amount of phosphopeptide present in the sample. Roughly speaking, the wider the peak eluting from the LC column the more frequently a phosphopeptide is detected by LC MS/MS and hence the more phosphopeptide present in the sample (see FIG. 1C). For example, comparison of phosphopeptide numbers for c-Met with the levels of phosphorylated c-Met protein observed by western analysis are in good agreement (Gilchrist, A., Au, C. E., Hiding, J., Bell, A. W., Fernandez-Rodriguez, J., Lesimple, S., Nagaya, H., Roy, L., Gosline, S. J., Hallett, M., et al. (2006). Quantitative proteomics analysis of the secretory pathway. Cell 127, 1265-1281; Old, W. M., Meyer-Arendt, K., Aveline-Wolf, L., Pierce, K. G., Mendoza, A., Sevinsky, J. R., Resing, K. A., and Ahn, N. G. (2005). Comparison of label-free methods for quantifying human proteins by shotgun proteomics. Mol. Cell. Proteomics 4, 1487-1502; Zybailov, B., Coleman, M. K., Florens, L., and Washburn, M. P. (2005). Correlation of relative abundance ratios derived from peptide ion chromatograms and spectrum counting for quantitative proteomic analysis using stable isotope labeling. Anal. Chem. 77, 6218-6224) (see FIG. 1D). We found this approach preferable to other methods such as parent ion peak height because it allowed simplifying the analysis by combining all sites on a given protein.

We next compared the distribution of protein tyrosine phosphorylation in NSCLC cell lines and solid tumors based upon protein classification.

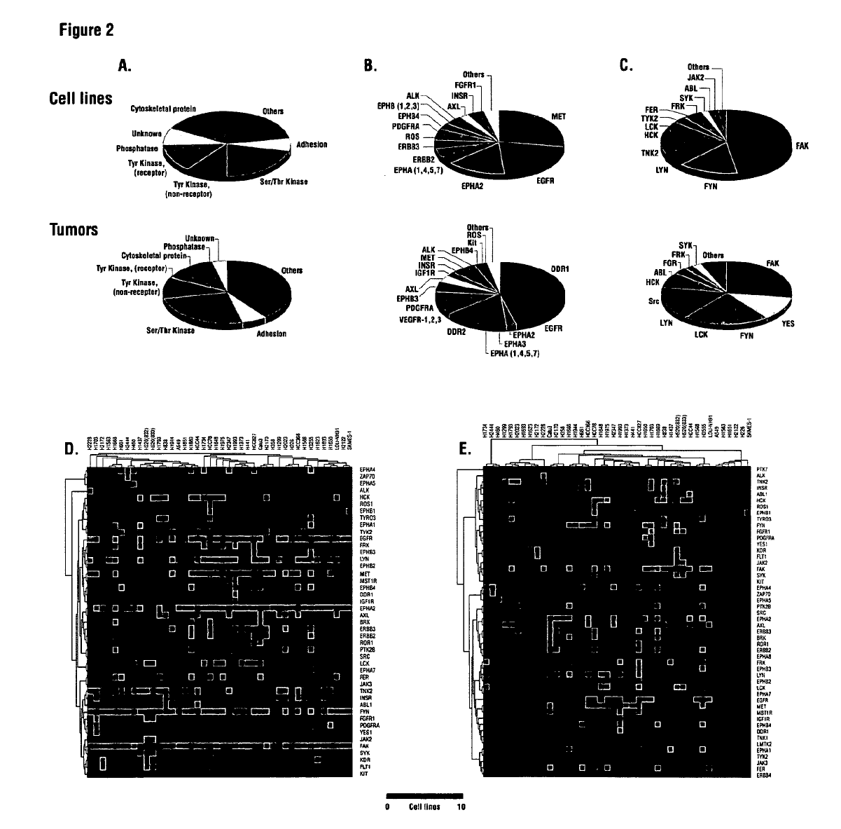

As shown in FIG. 2A, protein kinases, adhesion proteins, and components of the cytoskeleton were the most highly phosphorylated protein types. Tumors represent a complex tissue ranging from 50% to 90% cancer cells. The tyrosine kinases, c-Met, EGFR, and EphA2 showed the highest levels of receptor tyrosine kinase phosphorylation in cell lines while tumors showed high levels of DDR1, EGFR, DDR2, and Eph receptor tyrosine kinase phosphorylation (FIG. 2B). Fak and Src-family kinases made up the majority of NSCLC nonreceptor tyrosine kinase phosphorylation (FIG. 2C). Most phosphorylation occurred at the activation loop of these kinases. We analyzed 266 different phosphorylation sites on over 56 different tyrosine kinases and found that virtually all sites (with a few exceptions such as the src family C-terminal sites) were positively associated with kinase activity (Blume-Jensen, P., and Hunter, T. (2001). Oncogenic kinase signalling. Nature 411, 355-365; Ullrich, A., and Schlessinger, J. (1990). Signal transduction by receptors with tyrosine kinase activity. Cell 61, 203-212). Without wishing to be bound by any theory, we believe that tyrosine kinase phosphorylation is a good readout of kinase activity.

### Example 3

### Tyrosine Kinases Activated in NSCLC

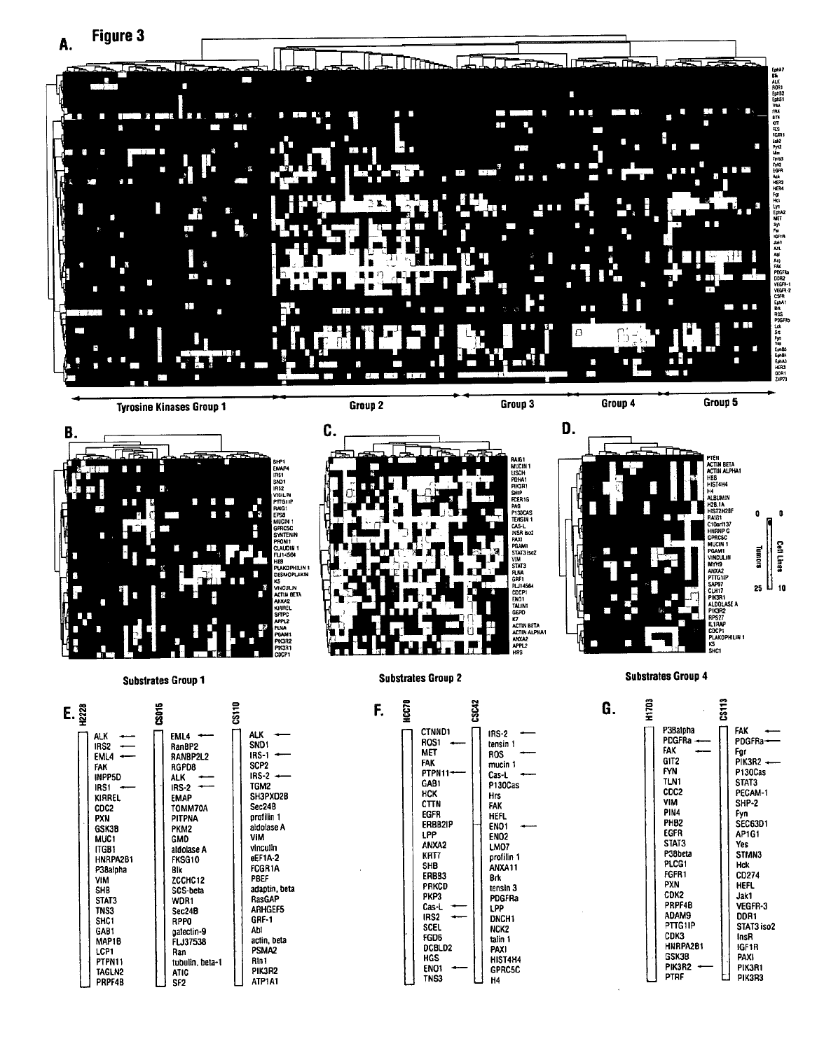

A fraction of NSCLC tumors and cell lines exhibited high tyrosine phosphorylation (FIGS. 1A and 1B) as a result of activated/overexpressed tyrosine kinases. To identify abnormally activated tyrosine kinases, we subtracted an average signaling profile derived from either the 41 different NSCLC cell lines or the 150 NSCLC tumors to obtain the unsupervised hierarchal clustering results shown in FIGS. 2E and 3A. This analysis highlighted differences among cell lines and identified highly phosphorylated (activated) tyrosine kinases (compare FIGS. 2D and 2E). Results were consistent with previous reports of activated EGFR (Amann, J., Kalyankrishna, S., Massion, P. P., Ohm, J. E., Girard, L., Shigematsu, H., Peyton, M., Juroske, D., Huang, Y., Stuart Salmon, J., et al. (2005). Aberrant epidermal growth factor receptor signaling and enhanced sensitivity to EGFR inhibitors in lung cancer. Cancer Res. 65, 226-235), ErbB2 (Stephens, P., Hunter, C., Bignell, G., Edkins, S., Davies, H., Teague, J., Stevens, C., O'Meara, S., Smith, R., Parker, A., et al. (2004). Lung cancer: intragenic ERBB2 kinase mutations in tumours. Nature 431, 525-526), ErbB3 (Engelman, J. A., Janne, P. A., Mermel, C., Pearlberg, J., Mukohara, T., Fleet, C., Cichowski, K., Johnson, B. E., and Cantley, L. C. (2005). ErbB-3 mediates phosphoinositide 3-kinase activity in gefitinib-sensitive nonsmall cell lung cancer cell lines. Proc. Natl. Acad. Sci. USA 102, 3788-3793), EphA2 (Kinch, M. S., Moore, M. B., and Harpole, D. H., Jr. (2003). Predictive value of the EphA2 receptor tyrosine kinase in lung cancer recurrence and survival. Clin. Cancer Res. 9, 613-618), and c-Met (Ma, P. C., Jagadeeswaran, R., Jagadeesh, S., Tretiakova, M. S., Nallasura, V., Fox, E. A., Hansen, M., Schaefer, E., Naoki, K., Lader, A., et al. (2005). Functional expression and mutations of c-Met and its therapeutic inhibition with SUI 1274 and small interfering RNA in nonsmall cell lung cancer. Cancer Res. 65, 1479-1488) receptor tyrosine kinases in NSCLC cell lines. EGFR kinase activity was elevated in 11 cell lines (FIG. 2E), and among these, five cell lines harbor EGFR-activating mutations. For example, we observed high levels of EGFR phosphopeptides in HCC827 (Amann, J., Kalyankrishna, S., Massion, P. P., Ohm, J. E., Girard, L., Shigematsu, H., Peyton, M., Juroske, D., Huang, Y., Stuart Salmon, J., et al. (2005). Aberrant epidermal growth factor receptor signaling and enhanced sensitivity to EGFR inhibitors in lung cancer. Cancer Res. 65, 226-235) and H3255 (Paez, J. G., Janne, P. A., Lee, J. C., Tracy, S., Greulich, H., Gabriel, S., Herman, P., Kaye, F. J., Lindeman, N., Boggon, T. J., et al. (2004). EGFR mutations in lung cancer: correlation with clinical response to gefitinib therapy. Science 304, 1497-1500; Tracy, S., Mukohara, T., Hansen, M., Meyerson, M., Johnson, B. E., and Janne, P. A. (2004). Gefitinib induces apoptosis in the EGFRL858R non-small-cell lung cancer cell line H3255. Cancer Res. 64, 7241-7244), known to express amplified and mutated EGFR. We observed high levels of c-Met and ErbB2 in H1993 and Calu-3 cell lines, respectively, consistent with previous reports (Lutterbach, B., Zeng, Q., Davis, L. J., Hatch, H., Hang, G., Kohl, N. E., Gibbs, J. B., and Pan, B. S. (2007). Lung cancer cell lines harboring MET gene amplification are dependent on Met for growth and survival. Cancer Res. 67, 2081-2088; Ma, P. C., Jagadeeswaran, R., Jagadeesh, S., Tretiakova, M. S., Nallasura, V., Fox, E. A., Hansen, M., Schaefer, E., Naoki, K., Lader, A., et al. (2005). Functional expression and mutations of c-Met and its therapeutic inhibition with SU11274 and small interfering RNA in nonsmall cell lung cancer. Cancer Res. 65, 1479-1488; Minami, Y., Shimamura, T., Shah, K., Laframboise, T., Glatt, K. A., Liniker, E., Borgman, C. L., Haringsma, H. J., Feng, W., Weir, B. A., et al. (2007). The major lung cancer-derived mutants of ERBB2 are oncogenic and are associated with sensitivity to the irreversible EGFR/ERBB2 inhibitor HK1-272. Oncogene 26, 5023-5027) and confirming known receptor tyrosine kinase activity in NSCLC cell lines.

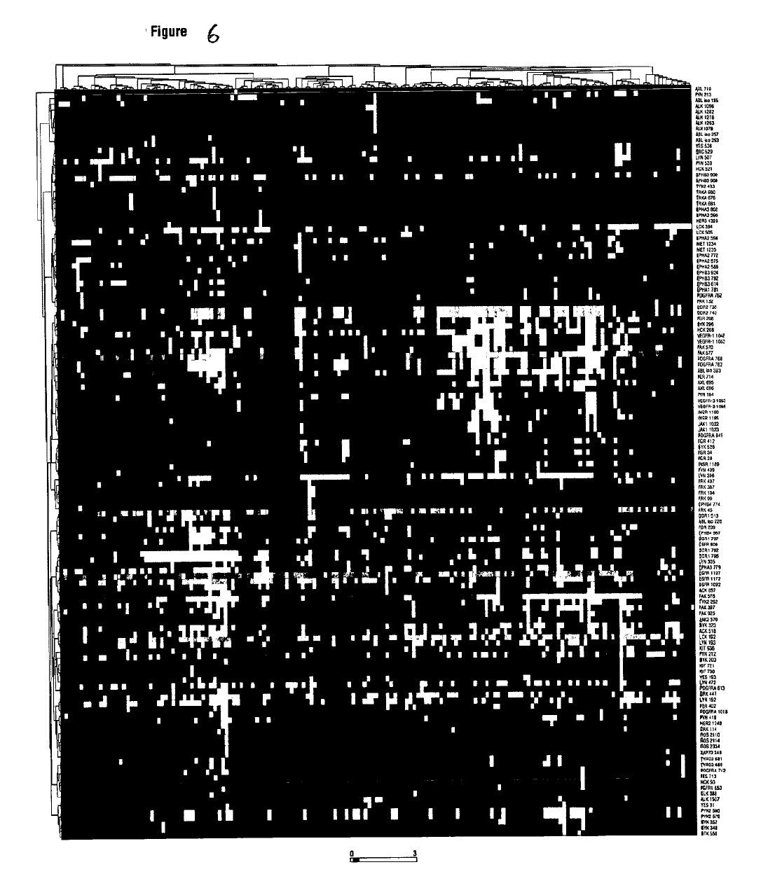

A similar analysis of NSCLC tumors is shown in FIG. 3A for all tyrosine kinases and in FIG. 6 for all tyrosine kinase phosphorylation sites. We identified five major groups of tumors using unsupervised Pearson clustering (FIG. 3A). From left to right are tumors aberrantly expressing the following: only one or two highly active tyrosine kinases (group 1), tumors expressing active Fak together with many different Src, Abl, and receptor tyrosine kinases (group 2), tumors expressing activated DDR1 together with src and abl kinases (group 3), tumors expressing Src kinases with RTKs such as EGFR (group 4), and tumors expressing predominately src and Abl tyrosine kinases (group 5).

### Example 4

### Tyrosine Kinase Substrate

We separated the analyzed phosphorylated substrates (excluding tyrosine and Ser/Thr kinases) from each group described in EXAMPLE 3. We identified the 30 most informative substrates (from over 2500 phosphorylated proteins) for groups 1, 2, and 4 (FIGS. 3B-3D). The different groups have different active kinases and different phosphorylated substrates. Group 2 tumors, with many active tyrosine kinases, showed higher levels of downstream phosphorylation than group 1 tumors. For example, group 2 tumors showed phosphorylation of proteins involved in motility and cytoskeleton dynamics as well as cell-surface receptors and glycolytic enzymes. Overall, group 1 tumors expressed lower levels of substrate phosphorylation that fall into several subgroups showing high SHP-1, IRS-1/2, and PI3KR1/2. Group 4 tumors showed phosphorylation of different substrates including PTEN and histones.

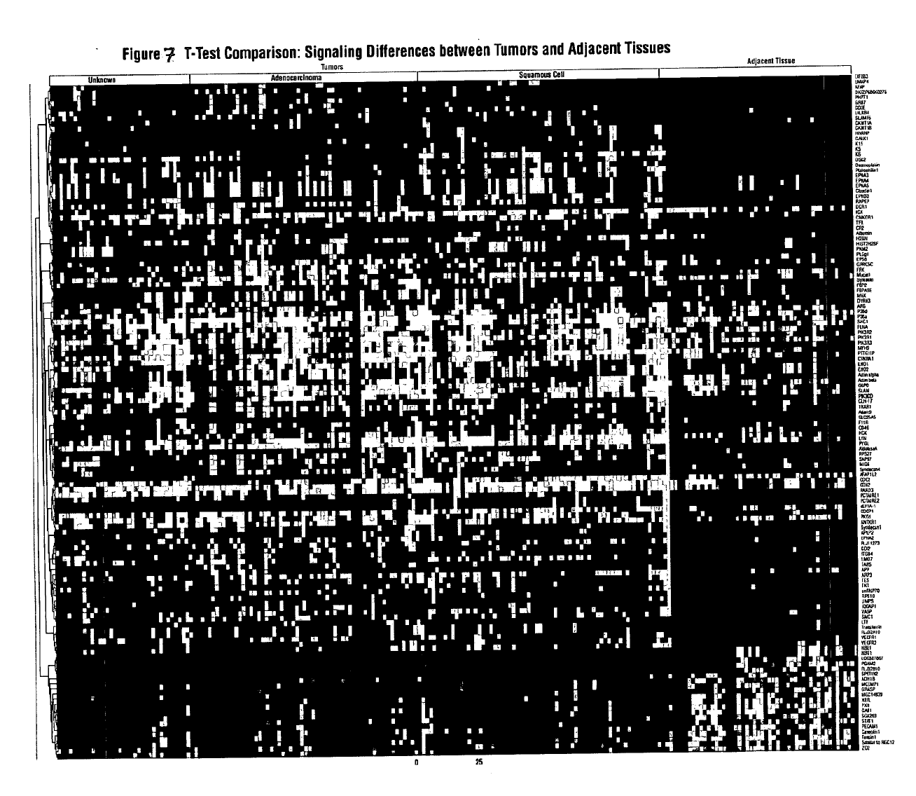

In general, we observed high phosphotyrosine IHC staining for group 2 tumors, consistent with the MS/MS results. We found no striking correlations of hierarchal clustering groups with available patient clinical data and tumor pathology. We also compared tumor protein tyrosine phosphorylation to 48 adjacent lung tissue samples using t test comparison (FIG. 7). This analysis identified significant signaling differences between tumor and normal tissue, including many cytoskeleton and signaling proteins.

### Example 5

### Ranking Activated Tyrosine Kinases

We found that a fraction of cell lines and tumors expressed multiple activated tyrosine kinases (see group 2 tumors), complicating the identification of “driver” kinase(s) (causally related to disease pathogenesis) from other activated kinases functioning in downstream networks. In addition, we also found that hierarchical clustering was not useful in grouping tumors with high EGFR phosphorylation (see FIG. 3A). This prompted us to instead develop an approach to identify candidate driver tyrosine kinases based upon identifying unusually high levels of tyrosine kinase activity in a subgroup of patients. We summed total phosphorylation for each kinase across either FIG. 2E or FIG. 3A and divided it by the number of cell lines or patients showing above average phosphorylation. Table 1 shows the most highly phosphorylated receptor tyrosine kinases ranked by average phosphorylation/patient or cell line. This analysis identified unusually high tyrosine kinase phosphorylation in subsets of cell lines or patients. Of the top 20 RTKs, 15 were identified in both cell lines and tumors. Of the top 10, Met, ALK, ROS, PDGFRa, DDR1, and EGFR were found in both cell lines and tumors (Table 1).

TABLE 1
Comparison of RTK Phosphorylation in Subgroups of NSCLC Cell Lines and Tumors.

| | | | | NSCLC tumors | | | |
|---|---|---|---|---|---|---|---|
| NSCLC cell lines | | | | | Normalized | | |
| | Phospho- | Number | Phospho | | phospho- | Number | Phospho |
| | peptide | of cell | level/ | | peptides | of | level/ |
| RTK's | sum | lines | cell line | RTK's | sum | samples | sample |
| ROS | 43 | 1 | 43 | MET | 847 | 12 | 71 |
| ALK | 36 | 1 | 36 | ALK | 464 | 7 | 66 |
| MET | 233 | 11 | 21 | DDR1 | 3136 | 63 | 50 |
| PDGFRa | 40 | 2 | 20 | ROS | 50 | 1 | 50 |
| ErbB2 | 44 | 3 | 15 | VEGFR-2 | 662 | 16 | 41 |
| EGFR | 132 | 11 | 12 | IGF1R | 675 | 18 | 37 |
| DDR1 | 9 | 1 | 9 | PDGFRa | 1295 | 37 | 35 |
| EphB4 | 28 | 4 | 7 | VEGFR-1 | 912 | 28 | 33 |
| FGFR1 | 20 | 3 | 7 | EGFR | 1298 | 43 | 30 |
| EphA2 | 64 | 10 | 6 | Axl | 761 | 26 | 29 |
| ErbB3 | 38 | 6 | 6 | EphB2 | 58 | 2 | 29 |
| VEGFR-1 | 16 | 3 | 5 | EphA2 | 772 | 29 | 27 |
| EphB1 | 10 | 2 | 5 | DDR2 | 1439 | 58 | 25 |
| Axl | 24 | 6 | 4 | FGFR1 | 93 | 4 | 23 |
| EphA4 | 15 | 4 | 4 | EphB3 | 793 | 38 | 21 |
| EphA1 | 14 | 4 | 4 | Mer | 199 | 10 | 20 |
| EphA5 | 3 | 1 | 3 | Tyro3 | 167 | 10 | 17 |
| Tyro3 | 12 | 4 | 3 | EphB4 | 269 | 19 | 14 |
| EphB2 | 11 | 5 | 2 | ErbB2 | 60 | 5 | 12 |
| IGF1R | 3 | 2 | 2 | Kit | 147 | 14 | 11 |

Abbreviations: RTK, receptor tyrosine kinase; NSCLC, non-small cell lung cancer.
Identifying high kinase activity (phosphorylation) in subsets of cell lines and patients. For patient samples, phosphopeptide sum represents each protein's spectral counts normalized to those for GSK3 beta and summed across all 150 tumors, minus the average count for that protein over all tumors. Number of samples represents the number of tumors showing above average phosphopeptide count. For cell lines, phosphopeptide sum represents each protein's spectral counts after subtraction of the average count for that protein over all 41 cell lines; because the same number of cells was used in each experiment, normalization was omitted. Cell lines and tissues are ranked in order of decreasing counts per sample.

We next applied a ranking process to identify candidate disease drivers by ranking kinases based upon total phosphorylation. Among all cell lines with the highest EGFR rank, we found that EGFR was often the most highly phosphorylated tyrosine kinase, in others it is among the top 2 or 3 kinases. We found all 5 cell lines carrying known EGFR-activating mutations and cell lines carrying known EGFR genomic amplification among the cell lines with highest EGFR rank.

We performed a similar analysis of NSCLC tumor samples using phosphorylation rank to identify tumors showing activated EGFR (Table 2). NSCLC tumors in this study were all stage 1 or 2 and consist of 74% males, 52% smokers, and 30% adenocarcinoma. We found that, among the 18 tumors with highest EGFR rank, 16 gave readable EGFR kinase domain DNA sequence (Table 2); of these, 9/16 tumors showed kinase domain-activating mutations with 8/8 adenocarcinomas and 5/5 female nonsmokers showing EGFR-activating mutations, consistent with previous reports of enrichment for female nonsmokers and adenocarcinoma (Lynch, T. J., Bell, D. W., Sordella, R., Gurubhagavatula, S., Okimoto, R. A., Brannigan, B. W., Harris, P. L., Haserlat, S. M., Supko, J. G., Haluska, F. G., et al. (2004). Activating mutations in the epidermal growth factor receptor underlying responsiveness of non-small-cell lung cancer to gefitinib. N. Engl. J. Med. 350, 2129-2139; Pao, W., Miller, V., Zakowski, M., Doherty, J., Politi, K., Sarkaria, I., Singh, B., Heelan, R., Rusch, V., Fulton, L., et al. (2004). EGF receptor gene mutations are common in lung cancers from “never smokers” and are associated with sensitivity of tumors to gefitinib and erlotinib. Proc. Natl. Acad. Sci. USA 101, 13306-13311) (Table 2).

TABLE 2
Patients Grouped by Receptor Tyrosine Kinase Phosphorylation

| | EGFR | | | | | | | | ALK | | | | | | | |
|---|---|---|---|---|---|---|---|---|---|---|---|---|---|---|---|---|
| | n06- | bj- | n06- | no6- | no6- | cs- | n06- | no6- | cs- | n06- | cs- | n06- | cs- | n06- | n06- | n06- |
| Name | cs98-2 | 635 | bj669 | cs89 | cs91 | 041 | cs105 | bj5737 | 057 | cs23 | 133 | cs93-1 | 103 | bj505 | bj590 | cs75 |
| EGFR | 131 | 38 | 15 | 18 | 15 | 35 | 56 | 6 | 9 | 32 | 12 | 48 | 24 | 24 | 81 | 38 |
| DDR1 | 120 | | | 18 | 128 | 14 | | | 19 | | 30 | 95 | 31 | | 95 | |
| IGF1R | 69 | | | | | | 44 | | | | | | | | | |
| VEGFR-1 | 43 | | | 12 | 27 | | | | 2 | | | 6 | 8 | | | |
| PDGFRa | 38 | 17 | | 24 | 21 | | | | | | 11 | | 24 | | 38 | |
| DDR2 | 33 | 12 | 16 | | 50 | | | 8 | | | 14 | 16 | 33 | 12 | 33 | |
| EphA3 | | 11 | | | | 20 | | | | | 4 | | | | | |
| InsR | 70 | | | 13 | 11 | | | | | | | | | | | |
| EphB3 | | | | | | 1 | | | 2 | 6 | 1 | 43 | | | 18 | |
| EphA2 | | | | | 27 | 2 | | | | | | | | | 19 | |
| Axl | 42 | 64 | | | | | | | | | | 9 | | | | |
| MET | | 8 | | 39 | | | | | | | | | | | | |
| VEGFR-2 | | | | 14 | 62 | | | | | | | | | | | |
| | del- | | del- | L747_{—} | del- | | | del- | E746_{—} | | | | | | | |
| EGFR | E746- | | E746- | A750 > | E746- | L833V: | | E746- | T751 > | | | | | | | |
| mutation | A750 | L858R | A750 | P | A750 | L838V | L858R | A750 | IP | wt | wt | wt | wt | wt | wt | wt |
| Pathology | AD | AD | AD | AD | AD | AD | AD | AD | SCC | | SCC | SCC | SCC | SCC | SCC | SCC |
| Smoking | no | no | no | no | yes | no | yes | yes | no | | yes | no | no | yes | no | no |
| Gender | F | F | F | F | M | M | M | M | F | M | M | M | M | M | M | M |

| | ROS | | | | | | | | | | | PDGFRa | | | | |
|---|---|---|---|---|---|---|---|---|---|---|---|---|---|---|---|---|
| | cs- | cs010/ | cs- | gz- | gz- | cs- | | cs- | | cs- | | cs- | cs- | cs- | bj- | cs- |
| Name | 015 | 011 | c45 | 30 | 70 | 110 | Name | 042 | Name | 97 | Name | 94 | 113 | 122 | 504 | 22 |
| ALK | 55 | 25 | 10 | 13 | 27 | 335 | ROS | 50 | Met | 727 | PDGFRa | 108 | 113 | 88 | 72 | 63 |
| DDR1 | 12 | 9 | | | 12 | | DDR1 | 12 | EphA2 | 194 | DDR2 | 23 | 33 | 33 | 16 | 2 |
| VEGFR1 | 17 | | | | | | PDGFRa | 17 | PDGFRa | 88 | VEGFR1 | 33 | | | | 18 |
| Mer | | 9 | | | | | Met | 7 | EGFR | 81 | DDR1 | 45 | 45 | 7 | 12 | |
| EphA3 | 8 | 4 | | | | | EphB3 | 6 | VEGFR2 | 62 | VEGFR2 | 36 | 46 | 46 | | |
| EGFR | 6 | 2 | | | 9 | 2 | ERBB2 | 4 | VEGFR1 | 60 | EGFR | 1 | 6 | 6 | | |
| EphB3 | | 22 | | | | | | | Axl | 59 | Axl | 72 | 17 | | | |
| Tyro3 | | 9 | | | | | | | | | | | | | | |
| EphB4 | | 3 | | | | | | | | | | | | | | |
| EphA2 | | | | | | 13 | | | | | | | | | | |
| ALK | EML4- | EML4- | EML4- | | | TFG- | ROS | CD74- | | | | | | | | |
| fusions | ALK | ALK | ALK | | | ALK | fusion | ROS | | | | | | | | |
| Pathology | SCC | AD | AD | | | AD | Pathology | AD | Pathology | SCC | Pathology | SCC | SCC | SCC | AD | AD |
| Smoking | no | no | no | | | no | Smoking | no | Smoking | yes | Smoking | yes | yes | yes | no | yes |
| Gender | M | F | M | | | M | Gender | F | Gender | M | Gender | M | M | M | F | M |

Abbreviations: AD, adenocarcinoma; SCC, squamous cell carcinoma
Patients grouped by high EGFR, Alk, Ros, Met and PDFGRa phosphorylation. For patient samples, each protein's spectral counts were normalized to those for GSK3 beta, and the average count for that protein over all tumors was subtracted. Above average receptor tyrosine kinase phosphorylation counts are shown. EGFR activating mutations, Alk and Ros transocations are indicated.

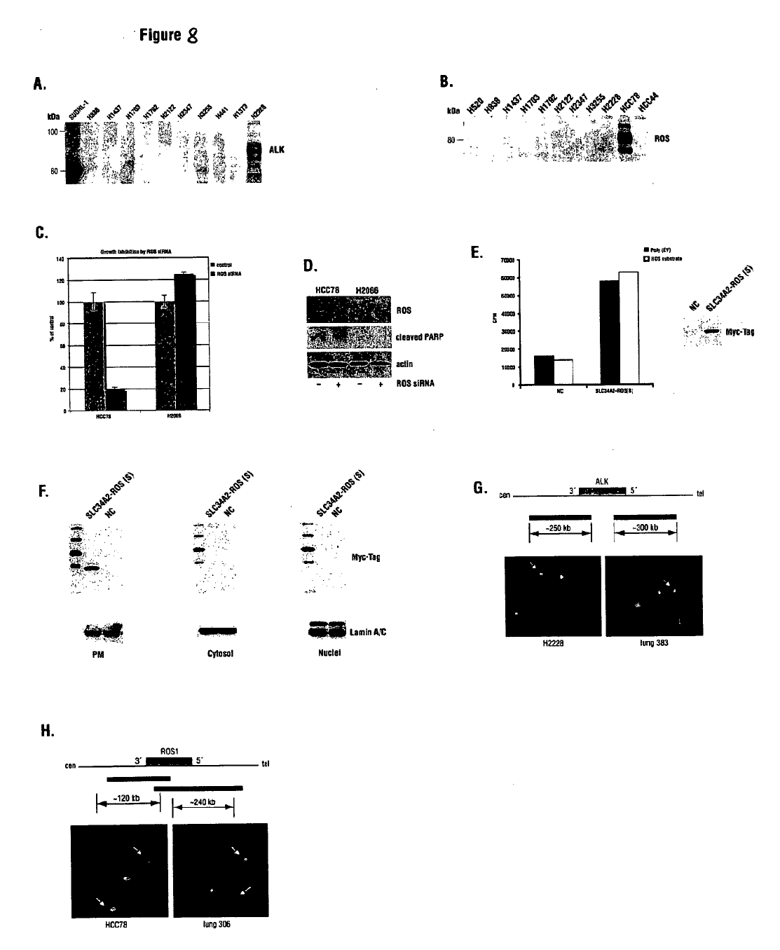

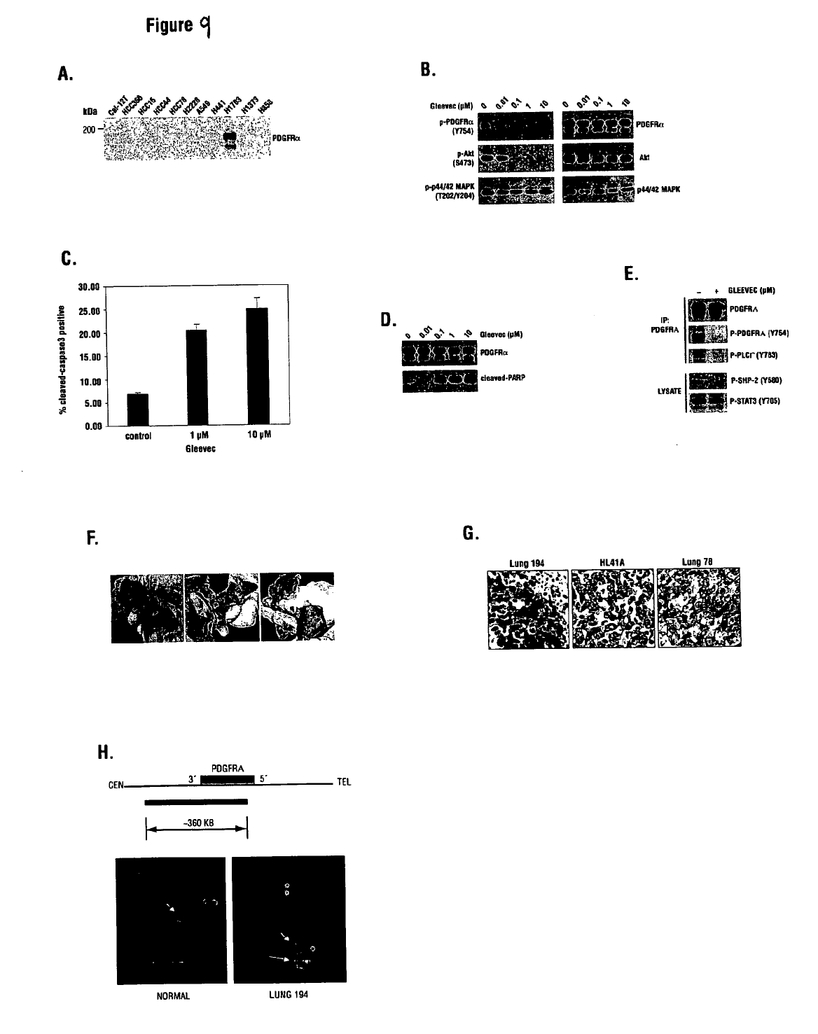

Having demonstrated that tumors with EGFR-activating mutations can be identified by EGFR phosphorylation rank, we applied the same approach to identify new candidate driver tyrosine kinases. As shown in Table 1, we found that Met, ALK, ROS, PDGFRa, DDR1, and EGFR were present in both cell lines and tumors. C-Met was found highly phosphorylated in one patient sample (Table 2), suggesting amplification as shown for H1993 cells where c-Met is a known driver (Lutterbach, B., Zeng, Q., Davis, L. J., Hatch, H., Hang, G., Kohl, N. E., Gibbs, J. B., and Pan, B. S. (2007). Lung cancer cell lines harboring MET gene amplification are dependent on Met for growth and survival. Cancer Res. 67, 2081-2088). In contrast to EGFR and c-Met, the kinases ALK, ROS, PDGFRa, and DDR1 have few literature connections to lung cancer. Because cell line models are critical to further testing the role of activated kinases in driving disease, we examined the expression of these candidates in NSCLC cell lines. Protein expression of ROS, ALK, and PDGFRa appeared to be highly upregulated in at least one NSCLC cell line (FIGS. 8A, 8B, and 9A). Although DDR1 is active in many tumors (Ford, C. E., Lau, S. K., Zhu, C. Q., Andersson, T., Tsao, M. S., and Vogel, W. F. (2007). Expression and mutation analysis of the discoidin domain receptors 1 and 2 in non-small cell lung carcinoma. Br. J. Cancer 96, 808-814), only H1993 cells express phosphorylated DDR1, and these cells are known to be driven by c-Met. Lack of a good DDR1 cell line model shifted the focus to ALK, c-ROS, and PDGFRα where MS/MS data identified corresponding NSCLC cell line models. Tables 2 shows cell lines and tumors expressing the highest levels of ALK, c-ROS, c-Met, and PDGFRα phosphorylation. As seen for EGFR, these RTKs are often but not always the most highly phosphorylated tyrosine kinase (Table 2), suggesting that they may play a role in driving disease. We also ranked all phosphorylated proteins for cell lines and selected tumors expressing ALK (FIG. 3E), c-ROS (FIG. 3F), and PDGFRa (FIG. 3G). Among the most highly phosphorylated substrates, many are shared between cell lines and tumors and may participate in downstream oncogenic signaling (see arrows FIGS. 3E-3G). We found phosphopeptides in HCC78, H2228, and H1703 cell lines and six different NSCLC tumors expressing ROS, ALK, EGFR, PDFGRalpha, and c-Met (over 2000 different phosphotyrosine sites).

We identified NSCLC tumors driven by EGFR-activating mutations. By ranking EGFR tyrosine kinase activity across cell lines and tumors, we found that high EGFR rank dramatically enriched for EGFR-activating mutations. Of 11 cell lines with high rank, 5 contained known EGFR-activating mutations, and of the 16 EGFR tumors from which we obtained sequence information, 8/9 were adenocarcinomas and 9 contained kinase domain-activating mutations. The remaining squamous cell carcinoma (SCC) patients showed high EGFR activity.

Roughly half of the high ranking EGFR cell lines and tumors carried EGFR-activating mutations. We thus grouped tumors based upon tyrosine kinase rank, leading to the identification of tumors expressing kinases activated above mean levels. We found the RTKs (Met, ALK, DDR1, ROS, VEGFR-2, IGF1R, PDGFRa, EGFR, and Ax1) and the non-RTKs (FAK, LYN, FYN, HCK, FRK, BRK, and others shown in FIG. 3A) to be highly phosphorylated in NSCLC.

### Example 6

### ALK and ROS Fusion Proteins in NSCLC Cell Lines and Tumors

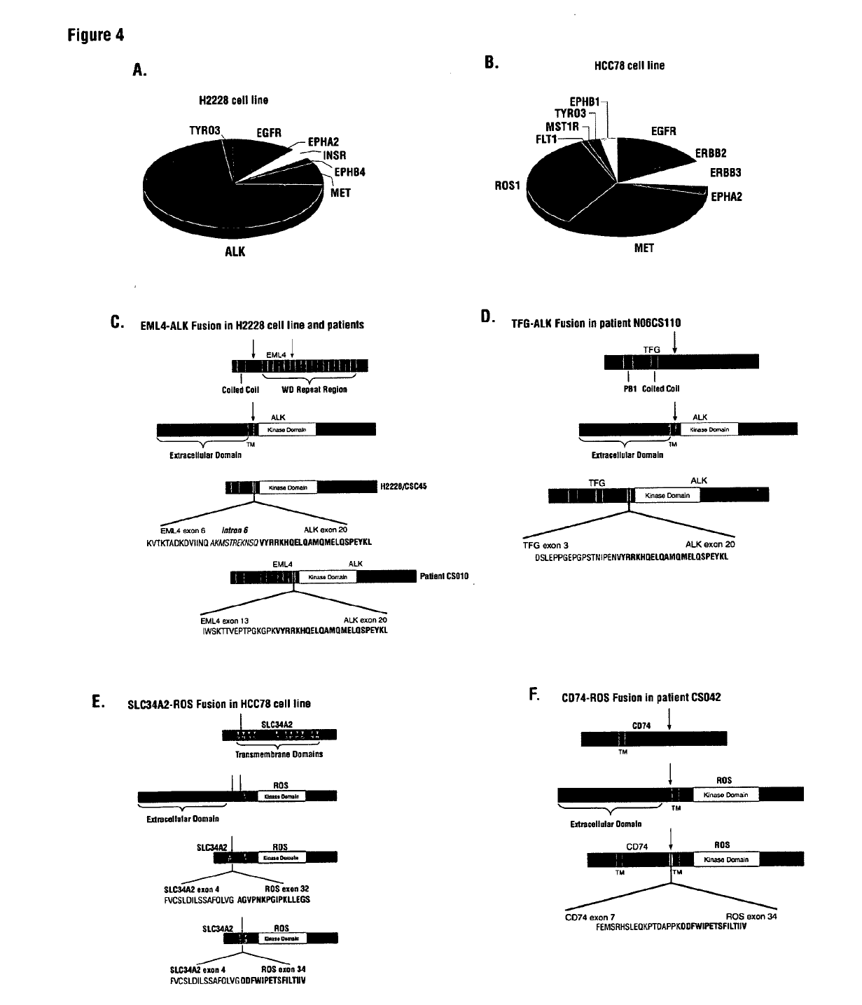

We observed high-level phosphorylation of ALK in the group of patients in the upper left corner of FIG. 3A, cell line H2228 (FIGS. 2E and 4A and Table 1) and ROS in one tumor sample and HCC78 cell line (FIG. 4B and Table 1). Phosphorylation rank place ALK and ROS near or at the top in these samples (Table 1). Protein expression of ALK and ROS was restricted among the NSCLC cell lines and exhibited a smaller than predicted molecular weight (FIGS. 8A and 8B). We performed RT-PCR and DNA sequencing to investigate the expressed RNA transcripts. 50 RACE analysis of RNA transcripts derived from H2228 cells and three different tumor samples demonstrated fusion of ALK to EML4, a microtubule-associated protein (see FIG. 4C). A short N-terminal region of EML4 was fused to the kinase domain of ALK at the precise point of fusion observed in other previously characterized ALK fusions (FIG. 4C), such as the NPM-ALK (Morris, S. W., Kirstein, M. N., Valentine, M. B., Dittmer, K. G., Shapiro, D. N., Saltman, D. L., and Look, A. T. (1994). Fusion of a kinase gene, ALK, to a nucleolar protein gene, NPM, in non-Hodgkin's lymphoma. Science 263, 1281-1284). ALK was also found fused to TFG (Hernandez, L., Pinyol, M., Hernandez, S., Bea, S., Pulford, K., Rosenwald, A., Lamant, L., Falini, B., Ott, G., Mason, D. Y., et al. (1999). TRK-fused gene (TFG) is a new partner of ALK in anaplastic large cell lymphoma producing two structurally different TFG-ALK translocations. Blood 94, 3265-3268) in one tumor sample (FIG. 4D). This fusion is the same as the short form of TFG-ALK previously observed (Hernandez, L., Bea, S., Bellosillo, B., Pinyol, M., Falini, B., Carbone, A., Ott, G., Rosenwald, A., Fernandez, A., Pulford, K., et al. (2002). Diversity of genomic breakpoints in TFG-ALK translocations in anaplastic large cell lymphomas: identification of a new TFG-ALK(XL) chimeric gene with transforming activity. Am. J. Pathol. 160, 1487-1494). In both EML4 and TFG fusions, a coiled-coil domain was fused to the kinase domain of ALK, likely conferring dimerization/oligomerization and constitutive kinase activity.

We performed a similar analysis of HCC78 cells and found fusion of ROS to the transmembrane solute carrier protein SLC34A2. The N-terminal region of SLC34A2, ending just after the first transmembrane region, was fused N-terminal to the transmembrane region of ROS producing a truncated fusion protein with two transmembrane domains. We observed two forms of this fusion protein in HCC78 cells that likely represent different splicing products produced from the same translocation event (see FIG. 4E). We identified a second ROS fusion in the c-ROS-positive NSCLC tumor. As shown in FIG. 4F c-ROS is fused to the N-terminal half of CD74, a type II transmembrane protein with high affinity for the MIF immune cytokine (Leng, L., Metz, C. N., Fang, Y., Xu, J., Donnelly, S., Baugh, J., Delohery, T., Chen, Y., Mitchell, R. A., and Bucala, R. (2003). MIF signal transduction initiated by binding to CD74. J. Exp. Med. 197, 1467-1476). The N-terminal region of CD74 was fused to ROS at the precise site of SLC34A2-ROS fusion (see FIG. 4E) creating a fusion protein with two transmembrane domains as found in the SLC34A2 fusion. Expression of a tagged SLC34A2-ROS fusion protein in mammalian cells showed constitutive kinase activity that localized to membrane fractions (see FIGS. 8E and 8F). We sequenced the kinase domains of ALK and ROS and found no mutations.

We found that experiments using siRNAs against ALK did not induce cell death in H2228 cells, suggesting survival signaling independent of ALK, such as activating mutations in PI3K (Samuels, Y., Diaz, L. A., Jr., Schmidt-Kittler, O., Cummins, J. M., Delong, L., Cheong, I., Rago, C., Huso, D. L., Lengauer, C., Kinzler, K. W., et al. (2005). Mutant P1K3CA promotes cell growth and invasion of human cancer cells. Cancer Cell 7, 561-573; Samuels, Y., and Velculescu, V. E. (2004). Oncogenic mutations of P1K3CA in human cancers. Cell Cycle 3, 1221-1224) or inactivation of PTEN (Mellinghoff, I. K., Wang, M. Y., Vivanco, I., Haas-Kogan, D. A., Zhu, S., Dia, E. Q., Lu, K. V., Yoshimoto, K., Huang, J. H., Chute, D. J., et al. (2005). Molecular determinants of the response of glioblastomas to EGFR kinase inhibitors. N. Engl. J. Med. 353, 2012-2024). We performed similar experiments using siRNAs against ROS. Two different siRNAs against ROS were effective in reducing ROS protein expression and inducing cell death in HCC78 cells (FIGS. 8C and 8D), demonstrating a strict dependence upon ROS signaling for HCC78 cell survival.

We analyzed the most highly phosphorylated substrates in ALK-expressing cell line and tumor samples (FIG. 3E) and identified candidate downstream signaling molecules such as SHIP2, IRS-1, and IRS-2 previously shown to be important downstream mediators of ALK signaling in anaplastic large cell lymphoma. In addition, phosphorylation of EML4, the fusion partner, was prominently seen (FIG. 3E). We identified PTPN11 and IRS-2 previously reported to be important downstream effectors of ROS in glioblastoma (Charest, A., Wilker, E. W., McLaughlin, M. E., Lane, K., Gowda, R., Coven, S., McMahon, K., Kovach, S., Feng, Y., Yaffe, M. B., et al. (2006). ROS fusion tyrosine kinase activates a SH2 domain-containing phosphatase-2/phosphatidylinositol 3-kinase/mammalian target of rapamycin signaling axis to form glioblastoma in mice. Cancer Res. 66, 7473-7481) as highly phosphorylated in c-ROS-expressing samples (FIG. 3F).

We prepared FISH break-apart probes to either side of the ALK or ROS locus and identified translocations in both c-ROS-expressing cell lines and tumors (FIG. 3H). As ALK and EML4 are located on the same arm of chromosome 2, deletion of the intervening DNA confirmed the expected break-apart pattern (FIG. 3G). We performed RT-PCR analysis using ALK and EML4 primers from 103 NSCLC tumors analyzed by MS/MS and identified 3 positive samples (Table 2) giving a 3% frequency for EML4-ALK; adding in the TGF-ALK sample gives an overall frequency of ALK fusions as 4% in the Chinese population.

### Example 7

### PDGFRα Activation in NSCLC: Sensitivity to Imatinib

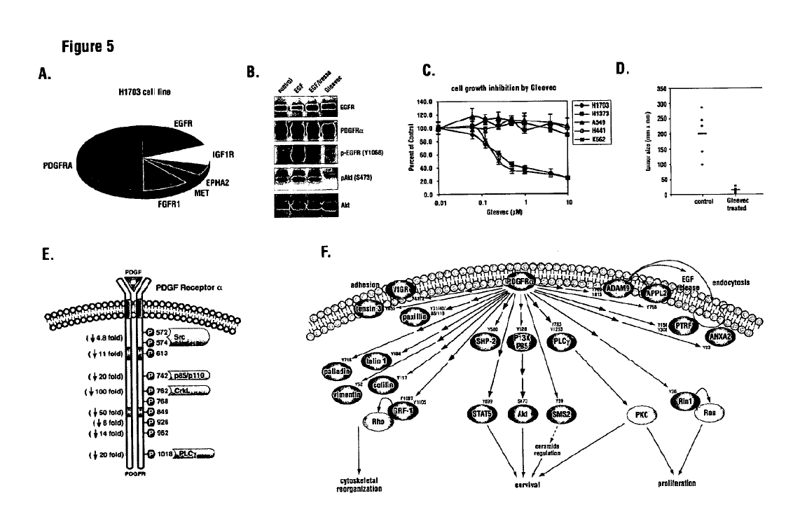

We identified PDGFRα as aberrantly activated in one NSCLC cell line, H1703, and eight different tumor samples (FIG. 5A and Table 1). We found that H1703 cells also express phosphorylated EGFR and FGFR1 and several other RTKs (FIG. 5A). We confirmed protein expression for PDGFRα by western blotting (FIG. 9A). We investigated sensitivity of H1703 cells to the PDGFR inhibitor Imatinib (Gleevec) and the EGFR inhibitor Gefitinib (Iressa). We found that phosphorylation of Akt at Ser473 was blocked by Imatinib but not by Gefitinib treatment (FIG. 5B). We also found that imatinib dose-response experiments (FIG. 9B) indicated almost complete inhibition of PDGFRα and Akt phosphorylation at 100 nM Imatinib with little if any effect on p44/42MAPK phosphorylation.

We performed cell proliferation MTT assays to further investigate the sensitivity of 20 NSCLC cell lines to Imatinib. As shown in FIG. 5C, H1703 cells showed a sensitivity profile similar to K562 cells that overexpress Bcr-Abl fusion protein (Druker, B. J., Sawyers, C. L., Kantarjian, H., Resta, D. J., Reese, S. F., Ford, J. M., Capdeville, R., and Talpaz, M. (2001). Activity of a specific inhibitor of the BCR-ABL tyrosine kinase in the blast crisis of chronic myeloid leukemia and acute lymphoblastic leukemia with the Philadelphia chromosome. N. Engl. J. Med. 344, 1038-1042; Mahon, F. X., Deininger, M. W., Schultheis, B., Chabrol, J., Reiffers, J., Goldman, J. M., and Melo, J. V. (2000). Selection and characterization of BCR-ABL positive cell lines with differential sensitivity to the tyrosine kinase inhibitor STI571: diverse mechanisms of resistance. Blood 96, 1070-1079). In contrast, 19 NSCLC cell lines (A549, H1373, H441, and many others negative for PDGFRa expression) were insensitive to Imatinib (FIG. 5C), correlating drug sensitivity with kinase phosphorylation. The observed Imatinib sensitivity profile differed from a previous report that identified PDGFRα expression in A549 cells and showed sensitivity to Imatinib (Zhang, P., Gao, W. V., Turner, S., and Ducatman, B. S. (2003). Gleevec (STI-571) inhibits lung cancer cell growth (A549) and potentiates the cisplatin effect in vitro. Mol. Cancer. 2, 1). To examine the effects of Imatinib on apoptosis, we treated H1703 cells with Imatinib and examined cleavage of PARP and caspase 3 by western blotting and flow cytometry, respectively. Imatinib (0.1 mM) significantly increased cleaved caspase 3 and cleaved PARP expression in H1703 cells (FIGS. 8C and 8D). We next examined the effects of Imatinib in vivo using mouse xenograft models. We injected nude mice subcutaneously with HI 703 cells and monitored tumor formation over a period of several weeks. Upon appearance of the first visible tumors, we treated the mice daily with Imatinib (50 mg/kg) or vehicle for a 2 week period. Imatinib-treated mice showed immediate and profound effects on tumor growth, while tumor growth continued in control mice (FIGS. 5D and 8F). We quantified tumor growth in control and Imatinib-treated animals (FIG. 5D), demonstrating exquisite sensitivity to Imatinib even in the complex tumor environment.

To analyze the effects of Imatinib on phosphotyrosine signaling, we grew H1703 cells in heavy and light amino acid-labeled media, treated with and without Imatinib, and analyzed phosphopeptides by mass spectrometry/SILAC (Everley, P. A., Bakalarski, C. E., Elias, J. E., Waghorne, C. G., Beausoleil, S. A., Gerber, S. A., Faherty, B. K., Zetter, B. R., and Gygi, S. P. (2006). Enhanced analysis of metastatic prostate cancer using stable isotopes and high mass accuracy instrumentation. J. Proteome Res. 5, 1224-1231; Ong, S. E., Blagoev, B., Kratchmarova, I., Kristensen, D. B., Steen, H., Pandey, A., and Mann, M. (2002). Stable isotope labeling by amino acids in cell culture, SILAC, as a simple and accurate approach to expression proteomics. Mol. Cell. Proteomics 1, 376-386). Some proteins and phosphorylation sites changed upon treatment with Imatinib. Treatment of H1703 cells with Imatinib had different effects on different sites of the PDGFRα receptor (FIG. 5E). Ten sites of tyrosine phosphorylation were observed and three new sites were identified (Tyr613, 926, and 962). Imatinib also suppressed tyrosine phosphorylation of a number of important downstream signaling proteins including phospholipase Cg 1, the regulatory subunit of PI3K, Stat5, and SHP-2 (see FIG. 5F). In addition, Imatinib suppressed tyrosinephosphorylation of proteins regulating the cytoskeleton and actin reorganization and signaling molecules involved in membrane recycling and endocytosis. We found the cell-surface metalloproteinase Adam 9 (Mazzocca, A., Coppari, R., De Franco, R., Cho, J. Y., Libermann, T. A., Pinzani, M., and Toker, A. (2005). A secreted form of ADAM9 promotes carcinoma invasion through tumor-stromal interactions. Cancer Res. 65, 4728-4738) known to liberate ligands for EGFR and FGFR (Peduto, L., Reuter, V. E., Shaffer, D. R., Scher, H. I., and Blobel, C. P. (2005). Critical function for ADAM9 in mouse prostate cancer. Cancer Res. 65, 9312-9319) to be highly phosphorylated in H1703 cells. Imatinib also inhibited phosphorylation of the ras effector Rinl (Hu, H., Bliss, J. M., Wang, Y., and Colicelli, J. (2005). RIN1 is an ABL tyrosine kinase activator and a regulator of epithelial-cell adhesion and migration. Curr. Biol. 15, 815-823) and inhibited phosphorylation of SMS2, an enzyme involved in ceramide synthesis (Taguchi, Y., Kondo, T., Watanabe, M., Miyaji, M., Umehara, H., Kozutsumi, Y., and Okazaki, T. (2004). Interleukin-2-induced survival of natural killer (NK) cells involving phosphatidylinositol-3 kinasedependent reduction of ceramide through acid sphingomyelinase, sphingomyelin synthase, and glucosylceramide synthase. Blood 104, 3285-3293). Western analysis confirmed selected SILAC results (FIG. 9E). We repeated this experiment on three different occasions with similar results.

### Example 8

### PDGFRα in NSCLC Tumor Samples

We analyzed peptides from five tumors with the highest levels of PDGFR phosphorylation in Table 2. We found that these tumors (group 2; FIG. 3A) also expressed FAK, Abl, DDR1/2, and VEGF1/2 in addition to many other active tyrosine kinases. Similar to H1703 cells, these NSCLC tumors also showed highly phosphorylated adhesion and cytoskeleton proteins (FIG. 3G), suggesting engagement of cell motility pathways. We performed an independent analysis by IHC using a PDGFRα-specific antibody to screen NSCLC tumor samples and identified strong PDGFRa staining in 2%-3% of patient samples (FIG. 9G). The results also differed from the report (Zhang, P., Gao, W. Y., Turner, S., and Ducatman, B. S. (2003). Gleevec (STI-571) inhibits lung cancer cell growth (A549) and potentiates the cisplatin effect in vitro. Mol. Cancer. 2, 1) that 100% of NSCLC adenocarcinomas express PDGFRα. We observed amplification at the PDGFRα locus by fluorescence in situ hybridization (FISH) analysis in one of the IHC-positive NSCLC samples (FIG. 9H).

In order that the experimental procedures described in the Examples be more fully understood, some materials and methods used in the Examples are set forth below. These materials and methods are for the purpose of illustration only and are not to be construed as limiting the scope of the invention in any way.

Cell Culture, Reagents, Western Blot, and Immunoprecipitation Analysis

We purchased cell culture reagents from Invitrogen. We obtained human NSCLC cell lines from American Type Culture Collection. We purchased ROS and phospho-PDGFRα antibodies from Santa Cruz, all other antibodies from Cell Signaling Technology (CST). We performed Western blot and Immunoprecipitation analyses following CST protocols.

We obtained human NSCLC cell lines H520, H838, H1437, H1563, H1568, H1792, H1944, H2170, H2172, HCC827, H2228, H2347, A549, H441, H1703, H1373, H358, H1993, Calu-3, H1648, H1975, H1666, H1869, H1650, H1734, H1793, H2023, H661, H2444, H1299, H1693, H226, H1623, H1651, H460, H2122, and SKMES-1 from American Type Culture Collection, and cultured the cells in RPMI 1640 medium with 10% FBS and adjusted to contain 2 mM L-glutamine, 1.5 g/L sodium bicarbonate, 4.5 g/L glucose, 10 mM HEPES, 1.0 mM sodium pyruvate, penicillin/streptomycin. We purchased NSCLC cell lines HCC78, Cal-12T, HCC366, HCC15, HCC44, and LOU-NH91 from DSMZ, and cultured them in RPMI 1640 containing 10% FBS and penicillin/streptomycin. We maintained cells in a 5% CO2 incubator at 37° C. For the immunoaffinity precipitation and immunoblot experiments, we grew cells to 80% confluence and then starved them in RPMI medium without FBS overnight before harvesting. We dissolved drugs (Iressa and Gleevec) in DMSO to yield 10 mM stock solution and stored at −20° C. We washed treated cells twice with cold PBS and then lysed them in 1× cell lysis buffer (20 mM Tris-HCl, pH 7.5, 150 mM NaCl, 1 mM Na2EDTA, 1 mM EGTA, 1% Triton, 2.5 mM sodium pyrophosphate, 1 mM beta-glycerophosphate, 1 mM Na3VO4, 1 μg/ml leupeptin) supplemented with Complete, Mini, EDTA-free protease inhibitor cocktail (Roche). We sonicated lysates and centrifuged them at 14000 rpm for 15 min. We measured the protein concentration using Coomassie protein assay reagent (Pierce Chemical Co., Rockford, Ill.). We resolved equal amounts of total protein by 8-10% SDS-PAGE gel and transferred them to nitrocellulose membranes. We incubated blots overnight at 4° C. with the appropriate antibodies by following CST protocols. We used 500 ug of protein lysate for immunoprecipitation. We rocked the cleared protein lysate with 2 ug of proper antibody and 15 ul protein G agarose beads (Pierce) overnight at 4° C. We washed the beads three times with 1× cell lysis buffer and boiled them in 30 ul of 2×SDS-PAGE sample buffer for 5 min. We then analyzed bound protein by Western blot.

### Phosphopeptide Immunoprecipitation and Analysis by LC-MS/MS Mass Spectrometry

We performed phosphopeptide immunoprecipitation from different cell lines as described previously (Rush, J., Moritz, A., Lee, K. A., Guo, A., Goss, V. L., Spek, E. J., Zhang, H., Zha, X. M., Polakiewicz, R. D., and Comb, M. J. (2005). Immunoaffinity profiling of tyrosine phosphorylation in cancer cells. Nat. Biotechnol. 23, 94-101) using the PhosphoScan Kit (P-Tyr-100) from CST. Briefly, we lysed 100 million cells in urea lysis buffer (20 mM Hepes, pH 8.0, 9 M Urea, 1 mM sodium vanadate, 2.5 mM sodium pyrophosphate, 1 mM beta-glycerophosphate).

For tumor samples, we homogenized 200-500 mg tissue in urea lysis buffer (1 ml/100 mg tissue) using an electronic homogenizer PolyTron for 2 pulses of 30 seconds each time. We sonicated the lysate and cleared it by centrifugation. We reduced cleared lysate by DTT and alkylated it with iodoacetamide. We then diluted samples 4 times with 20 mM Hepes to reduce Urea concentration to 2M, and digested them by trypsin overnight at room temperature with gentle shaking. We cruedly purified peptides with Sep-Pak C18 cartridges. We lyophilized eluate and dissolved dried peptides in 1.4 ml of MOPS IP buffer (50 mM MOPS/NaOH pH 7.2, 10 mM Na₂PO₄, 50 mM NaCl) and removed insoluble material by centrifugation. We carried out immunoprecipitation at 4° C. for overnight with 160 ug phospho-tyrosine 100 antibody (CST) coupled to protein G agarose beads (Roche). We then washed the beads 3 times with 1 ml MOPS IP buffer and twice with 1 ml cold HPLC grade dH₂O in the cold. We concentrated peptides in the IAP eluate and further purified them on 0.2 μl reverse-phase StageTips (Rappsilber, J., Ishihama, Y., and Mann, M. (2003) Stop and go extraction tips for matrix-assisted laser desorption/ionization, nanoelectrospray, and LC/MS sample pretreatment in proteomics. Anal Chem. 75(3):663-70). We eluted peptides from StageTips with 5 μl of 60% MeCN, 0.1% TFA into an LC-MS sample vial and took them to dryness with a vacuum concentrator. We dissolved dry samples in 5 μl of 5% formic acid, 5% MeCN. We loaded the sample (4 μl) onto a 10 cm×75 μm PicoFrit capillary column (New Objective) packed with Magic C18 AQ reversed-phase resin (Michrom Bioresources) using a Famos autosampler with an inert sample injection valve (Dionex). We then developed the column with a 45-min linear gradient of acetonitrile in 0.4% acetic acid, 0.005% HFBA delivered at 280 nl/min (Ultimate, Dionex). We collected tandem mass spectra in a data-dependent manner with an LTQ ion trap mass spectrometer (ThermoFinnigan), using a top-ten method, a dynamic exclusion repeat count of 1, and a repeat duration of 30 sec. We collected samples which we ran on the LTQ-Orbitrap Tandem mass spectra with an LTQ—Orbitrap hybrid mass spectrometer, using a top-ten method, a dynamic exclusion repeat count of 1, and a repeat duration of 30 sec. We collected MS spectra in the Orbitrap component of the mass spectrometer and collected MS/MS spectra in the LTQ.

SILAC Analsysi of H1703 Cells Treated with Gleevec

We split equal number of H1703 cells and grew them in either light or heavy SILAC medium (RPMI medium lacking arginine and lysine supplemented with either regular L-Lysine:HCl and L-Arginine:HCL (Sigma) for light medium, or supplemented with L-arginine:HCl (U-13 C6,98%) and L-lysine:2HCl (U-13C6,98%; U-15N2,98%) (Cambridge Isotope Laboratories) for heavy medium. The medium also contained 10% FBS, and penicillin/streptomycin. We grew cells for at least five generations to reach 100 million cells in each medium type. We then treated cells grown in the heavy medium with 1 μM Gleevec for 3 hours. We lysed both treated and control cells in Urea lysis buffer and combined them for phosphopeptide immunoprecipitation experiment as described above.

Analysis of Phosphorylation Site Data Sets

To assign peptide sequences, we used the hash string-matching algorithm, implemented in Biofacet (Gene-IT) to search proteins in PhosphoSite. If the peptide sequence matched multiple proteins, the protein with the first accession number in alphabetical order was chosen as a representative. For example, GASQAGM#TGY*GMPR matches both SM22-alpha (P37802) and TAGLN3 (Q9U115) and would be assigned to SM22-alpha. For a few peptides, we mannually chose the best studied protein of a set to be the representative. In the case of the peptide GEPNVSY*ICSR matching both GSK3α (P49840) and GSK3β (P49841), we assigned GSK3β as the representative.

We counted the number of spectra observed for each peptide sequence in a mass spectrometry run (Liu, H., Sadygov, R. G., and Yates, J. R., 3rd. (2004). A model for random sampling and estimation of relative protein abundance in shotgun proteomics. Anal. Chem. 76, 4193-4201). We subjected spectra to the quality criteria described below (i.e., in “Methods for LTQ-FT MS, Sequest Searches and Vista (pTyr SILAC Samples)”). To calculate a protein spectrum count, we summed the numbers for all of the peptides assigned to each protein in that run. We carried out hierarchal clustering using TIGR's MeV program (Saeed, A. I., Sharov, V., White, J., Li, J., Liang, W., Bhagabati, N., Braisted, J., Klapa, M., Currier, T., Thiagarajan, M., et al. (2003) TM4: a free, open-source system for microarray data management and analysis. Biotechniques 34, 374-378) with Pearson Correlation Distance and Average linkage clustering. We imported the number of times a given phosphoprotein was identified (sum of all observed spectra assigned to that protein) into MeV and used it to assemble heat maps.

For each patient sample, we normalized each protein's spectral counts to those for GSK3β, and subtracted the average count for that protein over all tumors.

Methods for LTQ-FT MS Sequest Searches and Vista (pTyr SILAC Samples)

We LC-MS analyzed each phosphopeptide sample in duplicate. We packed a fused silica microcapillary column (125 μm×18 cm) with C18 reverse-phase resin (Magic C18AQ, 5 μm particles, 200 Å pore size, Michrom Bioresources, Auburn, Calif.). We loaded samples (4 μL) onto this column with an autosampler (LC Packings Famos, San Francisco, Calif.) and eluted them into the mass spectrometer by a 55-min linear gradient of 7 to 30% acetonitrile in 0.1% formic acid. We delivered the gradient at approximately 600 nl/min using a binary HPLC pump (Agilent 1100, Palo Alto, Calif.) with an in-line flow splitter. We mass analyzed eluting peptide ions with a hybrid linear ion trap-7 Tesla ion cyclotron resonance Fourier transform instrument (LTQ-FT, Thermo Electron, San Jose, Calif.). We employed a top-seven method, whereby we collected 7 data-dependent MS/MS scans in the linear ion trap based on measurements made during the previous MS survey scan in the ICR cell, with the linear ion trap and the Fourier transform instrument operating concurrently. We performed MS scans at 375-1800 m/z with an automatic gain control (AGC) target of 3×10⁶ and a mass resolution of 10⁵. For MS/MS the AGC was 4000, the dynamic exclusion time was 25 s, and singly-charged ions were rejected by charge-state screening.

We assigned peptide sequences to MS/MS spectra using Sequest software (v.27, rev.12) and a composite forward/reverse IPI human protein database. Search parameters were: trypsin as protease; 1.08 Da precursor mass tolerance; static modification on cysteine (+57.02146, carboxamidomethylation); and dynamic modifications on serine, threonine and tyrosine (+79.96633 Da, phosphorylation), lysine (+8.01420, ¹³C₆ ¹⁵N₂), arginine (+6.02013, ¹³C₆) and methionine (+15.99491, oxidation). We used a target/decoy database approach to establish appropriate score-filtering criteria such that the estimated false-positive assignment rate was <1%. In addition to exceeding charge-dependent XCorr thresholds (for z=2, XCorr≧2.2; for z=3, XCorr≧3.3; for z=4, XCorr≧3.5), we required assignments to contain phosphotyrosine, to have a mass accuracy of −5 to +25 ppm, and to contain either all-light or all-heavy lysine/arginine residues. We further evaluated assignments passing these criteria using a custom quantification program Vista (Bakalarski, C. E., Elias, J. E., Villen, J. Haas, W., Gerber, S. A., Everley, P. A., and Gygi, S. P. (2008) The Impact of Peptide Abundance and Dynamic Range on Stable-Isotope-Based Quantitative Proteomic Analyses. J. Proteome Res. 10.1021/pr800333e) to calculate peak areas and ultimately a relative abundance between heavy and light forms of each peptide. We did not consider identified peptides with signal-to-noise in the MS scan below 15 for quantification. For those peptides found only in one of the conditions we used the signal-to-noise ratio instead.

### 5′ RACE and RT-PCR

We performed rapid amplification of cDNA ends with the use of 5′ RACE system (Invitrogen). We extracted total RNA from cell lines and patients with RNeasy mini Kit (Qiagen). The primers used to identify aberrant Alk transcript in cell line and patients in 5′ RACE reaction are Alk-GSP1 primer (5′-GCAGTAGTTGGGGTTGTAGTC) for cDNA synthesis and Alk-GSP2 (5′-GCGGAGCTTGCTCAGCTTGT) and Alk-GSP3 (5′-TGCAGCTCCTGGTGCTTCC) for a nested PCR reaction. The primers used to identify aberrant Ros transcript in cell line and patient in 5′ RACE reaction are Ros-GSP1 primer (5′-TGGAAACGAAGAACCGAGAAGGGT) for cDNA synthesis and Ros-GSP2 (5′-AAGACAAAGAGTTGGCTGAGCTGCG) and Ros-GSP3 (5′-AATCCCACTGACCTTTGTCTGGCAT) for the nested PCR reaction. We purified the PCR product with PCR purification kit (Qiagen) and sequenced it using Alk-GSP3 and Ros-GSP3 respectively using ABI 3130 capillary automatic DNA sequencer (Applied biosystem).

SiRNA

We obtained the following ROS siRNA oligonucleoties from Proligo: ROS1 (6318-6340) 5′-AAGCCCGGAUGGCAACGUUTT-3′, ROS1 (7181-7203) 5′-AAGCCUGAAGGCCUGAACUTT-3′. We seeded NSCLC cells in 12 well plates the day before the transfection, transfected 100 nM ROS1 siRNA using Mirus TransIT-TKO Transfection Reagent and 48 hours after transfection serum starved cells for additional 24 hours. We harvested cells by trypsinization, counted them, and prepared cell lysate to examine ROS protein levels by western blotting.

Animal Studies

We purchased four to six weeks female NCR nude mice from Taconic ande used them to generate H1703 xenograft. We carried out experiments under an IACUC approved protocol. We followed institutional guidelines for the proper and humane use of animals in research. We generated tumors by injecting 10 mice with 5×10⁶H1703 cells and reconstituted basement membrane Matrigel (BD Biosciences) with 1:1 ratio in PBS. Drug treatment started when the tumor was about 1 mm×1 mm size. 5 mice were treated with Gleevec at 50 mg/kg/day by oral gavage using a ball ended feeding needle. 5 mice were untreated. We sacrificed animals 7 days after treatment initiation, and excised and weighed tumors. We measured the average tumor diameter using caliper in both control and treated groups of mice.

### Growth Inhibition Assay and Apoptosis Assay

We performed cell growth inhibition assay with CellTiter 96 Aqueous One Solution Cell Proliferation Assay (Promega) according to manufacturer's suggestion. Briefly, we seeded 1000 to 5000 cells onto flat-bottomed 96-well plates and grew them in complete medium with 10% FBS. After 24 hours, we changed the cell medium to 100 μl complete growth medium with 10% FBS containing various concentrations of Gleevec, and incubated the cells for an additional 72 hours. We applied each drug concentration to triplicate well of cells. At the end of the incubation, we added 20 μl of CellTiter 96 AQUESOUS One solution to each well, and incubated the plate for 1-4 hours. We read absorbance at 490 nm using a Titan Multiskan Ascent microplate reader (Titertek Instrument). We expressed growth inhibition as mean±SD value of percentage of absorbance reading from treated cells vs untreated cells. We repeated the assay at least three times. We calculated IC₅₀ with the use of OriginPro 6.1 software (OriginLab, Northampton, Mass.).

We measured Gleevec-induced apoptosis by quantifying caspase activation using flow cytometry. We treated cells with Gleevec (1 μM, 10 μM, or DMSO only) for 24 hrs in 15 cm triplicate plates. We rinsed cells briefly in PBS, gently scraped them off the dish in PBS with a cell scraper, pelleted them, and immediately fixed them with 3% formaldehyde in PBS for 10 min at 37° C. We then permeabilized the cells with ice-cold 90% methanol and stored them at −20° C. in this solution for further analysis. We aliquoted fixed and permeabilized cells (5×10⁶) into 12×75 mm polypropylene culture tubes, rinsed them in PBS by centrifugation, and then incubated them in PBS with 0.5% BSA (PBS/BSA) for 10 min at room temperature to block nonspecific binding. We then incubated cells with an AlexaFluor 488-conjugated cleaved caspase-3 (Asp 175) antibody (#9669, Cell Signaling Technology, Danvers, Mass.) diluted 1:10 in PBS/BSA for one hour at room temperature. We subsequently rinsed cells in PBS/BSA by centrifugation, resuspended them in 0.5 ml PBS/BSA, and analyzed them on a Beckman-Coulter FC500 flow cytometer using a 488 nm argon laser for excitation.

In Vitro Kinase Assay

We amplified the open reading frame of the short form of SLC34A2-ROS(S) fusion gene by PCR from cDNA of HCC78, and cloned it in frame to pExchange-2 vector (Strategene, Calif.) with C-terminal Myc-tag. We transfected 293T cells grown in DMEM with 10% fetal calf serum with pExchange-2 and pExchange-2/SLC34A2-ROS(S), respectively. We harvested cell lysates w 48 hour after transfection. Following immunoprecipitation with Myctag antibody, we washed Ros immune complex 3 times with kinase buffer (60 mM HEPES, 5 mM MgCl₂, 5 mM MnCl₂, 3 μM Na₃VO₄ and 2.5 mM DTT). We initiated kinase reactions by re-suspending the Ros immune complex into 50 μl kinase buffer that contains 25 μM ATP, 0.2 uCi/ul [gamma32p] ATP, with 1 mg/ml of either Poly (EY, 4:1) or AAAEEEYMMMFAKKK as substrate. We stopped reactions by spotting reaction cocktail onto p81 filter papers. We then washed samples and assayed them for kinase activity by detection with a scintillation counter.

Immunohistochemical Staining

We reviewed hematoxylin and eosin slides of NSCLCs for confirmation of histopathological diagnosis and selection of adequate specimens for tissue microarray (TMA) construction. We assembled TMAs using a Beecher tissue puncher/array system (Beecher Instruments). For each case, we acquired 3 core samples of tumor tissue from donor blocks. We cut serial 4-μm-thick tissue sections from TMAs for immunohistochemistry study. We stained initial sections for hematoxylin and eosin to verify histopathology. We deparafiinized the slides in xylene and rehydrated through a graded series of ethanol concentrations. We performed antigen retrieval (microwave boiling for 18 min in 0.01 M EDTA buffer). We blocked intrinsic peroxidase by 3% hydrogen peroxide for 10 min. We used 10% goat serum (Sigma) solution for blocking nonspecific antibody binding, and used the primary antibodies at the manufacturer recommended concentration. We left slides at 4° C. overnight. After removing the primary antibody by washing in TBST for 5 min three times, we incubated slides for 30 min with secondary antibody at room temperature. Following three additional washes in TBST, we visualized slides using streptavidin-biotinperoxidase. We scanned sections at low magnification. We estimated immunostaining score from 0-3 based on the percentage and intensity of stained tumor cells. We also recorded the distribution of staining, membrane or cytoplasmic, and assessed it at high magnification. We scored immunoreactivity semi-quantitatively by considering the percentage and intensity of the staining of the tumor cells. We also assessed the distribution of staining, membrane or cytoplasmic, at high magnification. We scored immunohistochemical staining visually a four-tiered scale (0 to 3). We considered samples with 5% of weakly stained cells to negative (score 0). We scored samples with >5 20% positive cells with weak staining intensity weakly positive (score 1). We scored samples with >20 50% of positive cells with moderate to strong staining moderate positive (score 2) and samples showing >50% of positive cells with strong intensity as strong positive (score 3). We considered NSCLC samples with IHC score 1 as positive samples.

Fluorescence In Situ Hybridization

We identified amplifications in the PDGFRα locus by FISH using a probe set that consists of two BAC clones spanning the PDGFRα locus (RP11-231C18, RP11-8OL11) and a centromere probe (CEP4, Vysis (Vysis, Dowers Grove, Ill., USA)). The centromere probe allows amplifications due to polysomy to be distinguished from amplifications of the PDGFRα locus itself. We labeled the PDGFRα probes with Spectrum Orange dUTP (Vysis), and CEP4 with Spectrum Green dUTP. For analyzing rearrangements involving ROS, we designed a dual color break-apart probe. We labeled a proximal probe (BAC clone RP1-179P9) and two distal probes (BAC clone RP11-323017, RP1-94G16) with Spectrum Orange dUTP or Spectrum Green dUTP, respectively. For ALK we obtained a dual color, break-apart rearrangement probe from Vysis (Vysis, Dowers Grove, Ill., USA). The break-apart rearrangement probes contain two differently labeled probes on opposite sides of the breakpoint of the ALK gene. For both the ROS and ALK probe sets, the native region will appear as an orange/green fusion signal when hybridized, while rearrangement at the locus will result in separate orange and green signals. We did labeling of the probes by nick translation and interphase FISH using formalin fixed paraffin embedded (FFPE) tissue sections according to the manufactures instructions (Vysis) with the following modifications. In brief, we re-hydrated paraffin embedded tissue sections and subjected them to microwave antigen retrieval in 0.01 M Citrate buffer (pH 6.0) for 11 minutes. We digested sections with Protease (4 mg/ml Pepsin, 2000-3000 U/mg) for 25 minutes at 37° C., dehydrated them and hybridized them with the FISH probe set at 37° C. for 18 hours. After washing, we applied 4′,6-diamidino-2-phenylindole (DAPI; 0.5 ug/ml) in Vectashield mounting medium (Vector Laboratories, Burlingame, Calif.) for nuclear counterstaining. We used arrays of 1 mm tissue cores from NSCLC patient samples for screening. We further analyzed positive samples using whole sections and counted at least 50 cells to analyze the frequency of cytogenetic changes. 18 patient samples were available from the set of PDGFRα IHC positive samples for screening with the FISH probe set. We scored 14 samples successfully and found one to contain a large amplification. The majority of the cancer cells contained the amplification. We analyzed H1703 xenografts but didn't find amplification.

### Accession Numbers

We deposited the nucleotide sequences of CD74-ROS: EU236945, SLC34A2-ROS (long): EU236946, SLC34A2-ROS (short): EU236947, EML4-ALK: EU236948 and protein sequences CD74/ROS: ABX59671, SLC34A2/ROS fusion protein long isoform: ABX59672, SLC34A2/ROS fusion protein short isoform: ABX59673, EML4/ALK: ABX59674 in GenBank.

1. A method of classifying cancer cells in a sample, comprising the steps of:
(a) obtaining a sample of cancer cells;
(b) detecting the presence, absence, or levels of one or more tyrosine kinases in at least one signaling pathway in the sample; and
(c) classifying the cancer cells based on the presence, absence, or levels of the one or more tyrosine kinases.

2. The method of claim 1, wherein step (b) comprises using one or more methods selected from the group consisting of FISH, IHC, PCR, MS, flow cytometry, Western blotting, and ELISA.

3. A method of classifying cancer cells in a sample, comprising the steps of:
(a) obtaining a sample of cancer cells;
(b) detecting the presence, absence, or levels of one or more phosphorylated tyrosine kinases in at least one signaling pathway in the sample; and
(c) classifying the cancer cells based on the presence, absence, or levels of one or more phosphorylated tyrosine kinases.

4. The method of claim 3, wherein step (b) comprises immunoprecipitating phosphopeptides and analyzing the immunoprecipitated phosphopeptides.

5. The method of claim 1, wherein the one or more tyrosine kinases is selected from the group consisting of EGFR, FAK, Src, ALK, PDGFRa, Erb2, ROS, cMet, Ax1, ephA2, DDR1, DDR2, and FGFR.

6. The method of claim 1, wherein step (c) comprises one or more statistical methods.

7. The method of claim 6, wherein step (c) comprises using unsupervised Pearson clustering.

8. The method of claim 3, wherein step (c) comprises classifying the cancer cells as having only one or two highly phosphorylated tyrosine kinases.

9. The method of claim 3, wherein step (c) comprises classifying the cancer cells as expressing phosphorylated Fak, Src, Abl, and at least one receptor tyrosine kinase selected from the group consisting of EGFR, ALK, PDGFRa, Erb2, ROS, cMet, Ax1, ephA2, DDR1, DDR2, FGFR, VEGR-2, IGFR1, LYN, HCK, HER2, IRS1, IRS2 and BRK.

10. The method of claim 3, wherein step (c) comprises classifying the cancer cells as expressing phosphorylated DDR1, Src, and Abl.

11. The method of claim 3, wherein step (c) comprises classifying the cancer cells as expressing phosphorylated Src and at least one receptor tyrosine kinases selected from the group consisting of EGFR, ALK, PDGFRa, Erb2, ROS, cMet, Ax1, ephA2, DDR1, DDR2, FGFR, VEGR-2, IGFR1, LYN, HCK, HER2, IRS1, IRS2 and BRK.

12. The method of claim 3, wherein step (c) comprises classifying the cancer cells as expressing phosphorylated Src and Abl.

13. The method of claim 1, wherein the cancer cells are from a cancer selected from the group consisting of lung cancer, hematological cancer, prostate cancer, breast cancer, and tumor of the gastrointestinal tract.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. A method of treating cancer in a subject, comprising the steps of:
(a) obtaining a sample of cancer cells from the subject;
(b) classifying the cancer cells based on the levels of one or more aberrantly expressed tyrosine kinases or one or more aberrantly phosphorylated tyrosine kinases in at least one signaling pathway in the sample; and
(c) administering an effective dose of one or more tyrosine kinase inhibitors based on the classification.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. The method of claim 3, wherein the one or more tyrosine kinases is selected from the group consisting of EGFR, FAK, Src, ALK, PDGFRa, Erb2, ROS, cMet, Ax1, ephA2, DDR1, DDR2, and FGFR.

53. The method of claim 3, wherein step (c) comprises one or more statistical methods.

54. The method of claim 3, wherein the cancer cells are from a cancer selected from the group consisting of lung cancer, hematological cancer, prostate cancer, breast cancer, and tumor of the gastrointestinal tract.

55. The method of claim 1, wherein the cancer cells are from a non-small cell lung cancer (NSCLC).

56. The method of claim 3, wherein the cancer cells are from a non-small cell lung cancer (NSCLC).

57. The method of claim 26, wherein the cancer is non-small cell lung cancer (NSCLC).


SEQUENCE LISTING (17 sequences)

SEQ ID NO: 1 - protein, 14 aa, Artificial Sequence
  Description of Artificial Sequence Synthetic peptide
GASQAGMTGYGMPR

SEQ ID NO: 2 - protein, 11 aa, Artificial Sequence
  Description of Artificial Sequence Synthetic peptide
GEPNVSYICSR

SEQ ID NO: 3 - DNA, 21 bp, Artificial Sequence
  Description of Artificial Sequence Synthetic primer
gcagtagttggggttgtagtc

SEQ ID NO: 4 - DNA, 20 bp, Artificial Sequence
  Description of Artificial Sequence Synthetic primer
gcggagcttgctcagcttgt

SEQ ID NO: 5 - DNA, 19 bp, Artificial Sequence
  Description of Artificial Sequence Synthetic primer
tgcagctcctggtgcttcc

SEQ ID NO: 6 - DNA, 24 bp, Artificial Sequence
  Description of Artificial Sequence Synthetic primer
tggaaacgaagaaccgagaagggt

SEQ ID NO: 7 - DNA, 25 bp, Artificial Sequence
  Description of Artificial Sequence Synthetic primer
aagacaaagagttggctgagctgcg

SEQ ID NO: 8 - DNA, 25 bp, Artificial Sequence
  Description of Artificial Sequence Synthetic primer
aatcccactgacctttgtctggcat

SEQ ID NO: 9 - DNA, 21 bp, Artificial Sequence
  Description of Combined DNA/RNA Molecule Synthetic oligonucleotide
aagcccggauggcaacguutt

SEQ ID NO: 10 - DNA, 21 bp, Artificial Sequence
  Description of Combined DNA/RNA Molecule Synthetic oligonucleotide
aagccugaaggccugaacutt

SEQ ID NO: 11 - protein, 15 aa, Artificial Sequence
  Description of Artificial Sequence Synthetic peptide
AAAEEEYMMMFAKKK

SEQ ID NO: 12 - protein, 48 aa, Artificial Sequence
  Description of Artificial Sequence Synthetic polypeptide
KVTKTADKDVIINQAKMSTREKNSQVYRRKHQELQAMQMELQSPEYKL

SEQ ID NO: 13 - protein, 39 aa, Artificial Sequence
  Description of Artificial Sequence Synthetic polypeptide
IWSKTTVEPTPGKGPKVYRRKHQELQAMQMELQSPEYKL

SEQ ID NO: 14 - protein, 41 aa, Artificial Sequence
  Description of Artificial Sequence Synthetic polypeptide
DSLEPPGEPGPSTNIPENVYRRKHQELQAMQMELQSPEYKL

SEQ ID NO: 15 - protein, 32 aa, Artificial Sequence
  Description of Artificial Sequence Synthetic polypeptide
FVCSLDILSSAFQLVGAGVPNKPGIPKLLEGS

SEQ ID NO: 16 - protein, 32 aa, Artificial Sequence
  Description of Artificial Sequence Synthetic polypeptide
FVCSLDILSSAFQLVGDDFWIPETSFILTIIV

SEQ ID NO: 17 - protein, 34 aa, Artificial Sequence
  Description of Artificial Sequence Synthetic polypeptide
FEMSRHSLEQKPTDAPPKDDFWIPETSFILTIIV